(19) United States
(12) Patent Application Publication
(10) Pub. No.: US 2026/0252577 A1
(43) Pub. Date: Aug. 27, 2026

(54) SYSTEM

(71) Applicant: SoftBank Group Corp., Tokyo (JP)

(72) Inventor: Minami NAKAMURA, Tokyo (JP)

(73) Assignee: SoftBank Group Corp., Tokyo (JP)

(21) Appl. No.: 19/541,412

(22) Filed: Feb. 17, 2026

(30) Foreign Application Priority Data
Feb. 21, 2025 (JP) 2025-027027

(51) Int. Cl. G06F 16/248; G06F 3/0482

(52) U.S. Cl. CPC G06F 16/248; G06F 3/0482

(57) ABSTRACT

The system according to the embodiment comprises a reception unit, a recording unit, a display unit, a reminder unit, and a suggestion unit. The reception unit is configured to receive voice input. The recording unit is configured to analyze the voice received by the reception unit and record it as text data. The display unit is configured to display the data recorded by the recording unit in chronological order. The reminder unit is configured to provide reminders for periodic examinations. The suggestion unit is configured to indicate body parts that have not been examined recently and propose human health checkup menus and nearby hospitals.

### CROSS-REFERENCE TO RELATED APPLICATION

The present application claims priority to and incorporates by reference the entire contents of Japanese Patent Application No. 2025-027027 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, the management of medical history and examination results is complicated, and there is a risk of missing the timing for periodic examinations.

### SUMMARY OF THE INVENTION

The system according to the embodiment comprises a reception unit, a recording unit, a display unit, a reminder unit, and a suggestion unit. The reception unit is configured to receive voice input. The recording unit is configured to analyze the voice received by the reception unit and record it as text data. The display unit is configured to display the data recorded by the recording unit in chronological order. The reminder unit is configured to provide reminders for periodic examinations. The suggestion unit is configured to indicate body parts that have not been examined recently and propose human health checkup menus and nearby hospitals.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

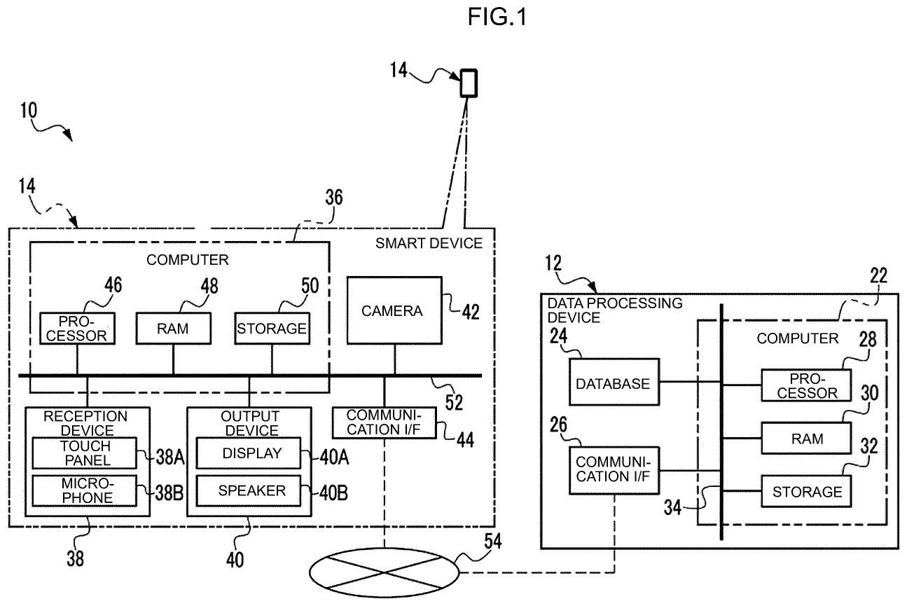

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

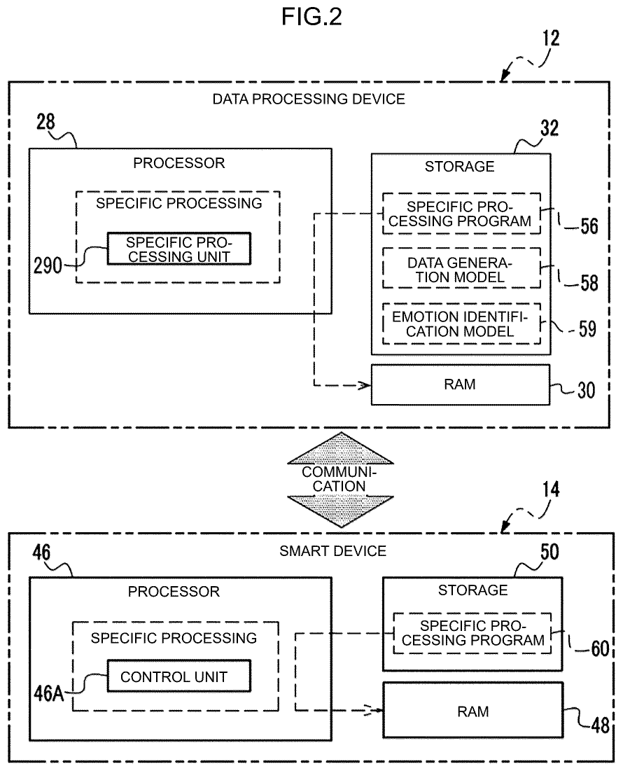

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The health management system according to the embodiment of the present invention is a system that records medical history and diagnostic results using voice input, and allows confirmation of medical history, medication history, and the timing and results of examinations in chronological order. This health management system receives voice input, which is analyzed by a generative AI and stored as text data. Furthermore, it provides reminders when periodic examinations are required, indicates body parts that have not been examined recently, and proposes appropriate human health checkup menus and nearby hospitals. For example, when a user speaks via voice about “which hospital and what diagnosis was given,” the system records the content. For instance, if the user says “I was diagnosed with gastritis at the hospital,” that information is recorded. This voice input is analyzed by the generative AI and stored as text data. Next, the user can confirm the medical history, medication history, and the timing and results of examinations in chronological order. For example, the user can view a list of which hospital provided which diagnosis in the past, which medication was taken and when, which examination was received and when, and what the results were. This information is analyzed by the generative AI and provided to the user. Furthermore, when the time for a periodic examination arrives, the system provides a reminder. For example, by providing a reminder such as “The next blood test is next month,” the system helps the user not forget the examination. This reminder function is also analyzed by the generative AI and notified at the appropriate timing. In addition, the system is equipped with a function to indicate body parts that have not been examined recently and to propose appropriate human health checkup menus and nearby hospitals. For example, it may propose, “You have not had a stomach examination recently, so we recommend a gastroscopy. You can receive it at a nearby hospital.” This proposal is also analyzed by the generative AI and provided to the user. Thus, the present invention is a system that records medical history and diagnostic results using voice input, allows confirmation of medical history, medication history, and the timing and results of examinations in chronological order, and supports the user's health management by providing reminders for periodic examinations and proposing appropriate examination menus and hospitals. As a result, the health management system can efficiently support the user's health management. Specifically, this health management system acquires one-dimensional chronological voice data sampled at 16 kHz (e.g., 5 seconds of PCM waveform data, length 80,000 samples) as voice input via the reception unit, performs preprocessing such as noise reduction and volume normalization, and then executes feature extraction processing such as spectrogram conversion and Mel-frequency cepstral coefficient (MFCC) extraction. Next, the recording unit inputs these feature vectors (e.g., 40-dimensional MFCC×100 frames) into a convolutional neural network (CNN), recurrent neural network (RNN), or Transformer-based speech recognition model to perform speech recognition and generate a string sequence (e.g., “I was diagnosed with gastritis at the hospital”) as output. Furthermore, a natural language processing module converts the obtained text data into structured data (e.g., JSON format {diagnosis: gastritis, hospital: XX, date: 2024-05-01}) by morphological analysis and named entity recognition (NER), and stores it in a database in chronological order. When the user checks the history, the display unit extracts the relevant records from the database and displays them as a list or graph in chronological order. The reminder unit scans the examination history in the database, determines the recommended interval for examinations (e.g., blood tests every 6 months) using rule-based or machine learning models (e.g., decision trees, random forests), and calculates the next scheduled examination date. The reminder unit also considers the user's calendar information and past examination tendencies to determine the optimal notification timing (e.g., one week before the scheduled examination date) and provides reminders via push notifications or email. The suggestion unit performs gap analysis of examination history (e.g., gastroscopy not performed in the past two years) and uses user attributes (age, gender, medical history) as input to generate suggestion sentences such as “gastroscopy recommended” using rule-based or generative AI (large language models). Furthermore, it links with a geographic information database to search for a list of nearby medical institutions based on the user's current location and incorporates this into the suggestion sentence. Examples of input to the AI include history data such as “diagnosed with gastritis on 2024-05-01,” “blood test on 2024-04-10,” “medication A taken on 2024-03-01,” and examples of AI output include “next blood test recommended on 2024-10-10,” “gastroscopy not performed,” “examination available at the nearest XX hospital.” These outputs are used for display and notification in the user interface, as well as input to subsequent health advice generation modules. As a technical effect, this system automatically generates structured data from voice input, greatly improving recording accuracy, searchability, and reminder accuracy compared to conventional manual input or paper records. In addition, history analysis by AI and individually optimized reminders and suggestions promote user health management behavior and reduce the risk of missed examinations and delayed treatment. Application fields include personal health management, corporate health management support, telemedicine services, and automation of records in nursing care settings. Furthermore, by anonymizing and aggregating data from multiple users, it can also be used for trend analysis in regional healthcare and public health policy planning.

The health management system according to the embodiment comprises a reception unit, a recording unit, a display unit, a reminder unit, and a suggestion unit. The reception unit is configured to receive content spoken by a user via voice. For example, when a user says “I was diagnosed with gastritis at the hospital,” the reception unit receives the content. The recording unit analyzes the voice received by the reception unit and records it as text data. For example, a generative AI converts the voice into text data using a speech recognition algorithm and records the content. The display unit displays the data recorded by the recording unit in chronological order. For example, the user can view a list of which hospital provided which diagnosis in the past, which medication was taken and when, which examination was received and when, and what the results were. The reminder unit provides reminders when periodic examinations are required. For example, by providing a reminder such as “The next blood test is next month,” the system helps the user not forget the examination. The suggestion unit indicates body parts that have not been examined recently and proposes appropriate human health checkup menus and nearby hospitals. For example, it may propose, “You have not had a stomach examination recently, so we recommend a gastroscopy. You can receive it at a nearby hospital.” As a result, the health management system according to the embodiment can efficiently support the user's health management. Specifically, this health management system acquires one-dimensional chronological voice data sampled at 16 kHz (e.g., 5 seconds of PCM waveform data, length 80,000 samples) as the reception unit using a high-sensitivity microphone or the voice input function of a smartphone. The reception unit automatically performs preprocessing such as noise reduction (spectral subtraction, Wiener filter, etc.) and volume normalization (peak normalization, RMS normalization, etc.) to improve the quality of the voice signal. The recording unit performs feature extraction processing such as spectrogram conversion and Mel-frequency cepstral coefficient (MFCC) extraction from the preprocessed voice data, and inputs the obtained feature vectors (e.g., 40-dimensional MFCC×100 frames) into a convolutional neural network (CNN), recurrent neural network (RNN), or Transformer-based speech recognition model. These models use pre-trained weights to generate string sequences (e.g., “I was diagnosed with gastritis at the hospital”) from voice with high accuracy and speed. The natural language processing module performs morphological analysis (e.g., MeCab) and named entity recognition (NER) on the obtained text data, and converts it into structured data such as “diagnosis name,” “hospital name,” and “date” (e.g., diagnosis: gastritis, hospital: XX, date: 2024-05-01). The recording unit stores these structured data in a database in chronological order, greatly improving the searchability and reusability of the data. The display unit extracts relevant records from the database on the user interface and displays them as a list or graph in chronological order. The reminder unit scans the examination history, determines the recommended interval for examinations (e.g., blood tests every 6 months) using rule-based or machine learning models such as decision trees and random forests, and calculates the next scheduled examination date. The reminder unit also considers the user's calendar information and past examination tendencies to determine the optimal notification timing (e.g., one week before the scheduled examination date) and provides reminders via push notifications or email. The suggestion unit performs gap analysis of examination history (e.g., gastroscopy not performed in the past two years) and uses user attributes (age, gender, medical history) as input to generate suggestion sentences such as “gastroscopy recommended” using rule-based or large language models. Furthermore, it links with a geographic information database to search for a list of nearby medical institutions based on the user's current location and incorporates this into the suggestion sentence. Examples of input to the AI include history data such as “diagnosed with gastritis on 2024-05-01,” “blood test on 2024-04-10,” “medication A taken on 2024-03-01,” and examples of AI output include “next blood test recommended on 2024-10-10,” “gastroscopy not performed,” “examination available at the nearest XX hospital.” These outputs are used for display and notification in the user interface, as well as input to subsequent health advice generation modules. As a technical effect, this system automatically generates structured data from voice input, greatly improving recording accuracy, searchability, and reminder accuracy compared to conventional manual input or paper records. In addition, history analysis by AI and individually optimized reminders and suggestions promote user health management behavior and reduce the risk of missed examinations and delayed treatment. Application fields include personal health management, corporate health management support, telemedicine services, and automation of records in nursing care settings. Furthermore, by anonymizing and aggregating data from multiple users, it can also be used for trend analysis in regional healthcare and public health policy planning.

The reception unit is capable of receiving content spoken by a user via voice. For example, when a user says “I was diagnosed with gastritis at the hospital,” the reception unit receives the content. The reception unit may use a microphone to receive voice input. Additionally, the reception unit may utilize the voice recognition function of a smartphone. Thus, the reception unit can receive content spoken by a user via voice. Specifically, the reception unit acquires one-dimensional chronological voice data sampled at 16 kHz (e.g., 5 seconds of PCM waveform data, length 80,000 samples) using a high-sensitivity microphone or the voice input function of a smartphone. The reception unit automatically performs noise reduction processing using spectral subtraction or Wiener filter, and volume normalization processing using peak normalization or RMS normalization on the acquired voice data to improve the quality of the voice signal. Furthermore, the reception unit may apply algorithms such as energy threshold determination or zero-crossing rate analysis to automatically detect the start and end timing of voice input. This enables the removal of unintended noise or silent sections and the extraction of only accurate voice segments. The reception unit may also be capable of simultaneously receiving input from multiple voice input devices (e.g., wearable microphones, Bluetooth headsets, in-vehicle microphones), and automatically selecting optimal preprocessing parameters for each device. Examples of input to the AI include natural spoken voice data such as “I was diagnosed with gastritis at the hospital” and “I jogged for 30 minutes today,” which are input as feature vectors after preprocessing to subsequent speech recognition models. As a technical effect, the reception unit can significantly improve subsequent speech recognition accuracy by automatically correcting for noise and volume variations in diverse voice input environments. Unlike conventional simple recording and playback methods, the reception unit provides high-quality voice data in real time, thereby improving the overall recording accuracy, usability, and response speed of the health management system. Application fields include personal health management, telemedicine, automation of records in nursing care settings, and corporate health management support.

The recording unit is capable of analyzing the voice received by the reception unit and recording it as text data. For example, the recording unit uses a generative AI to analyze the voice received by the reception unit and record it as text data. The generative AI converts the voice into text data using a speech recognition algorithm. For example, the generative AI analyzes the voice input and saves the content as text data. The text data is saved, for example, based on character encoding or database type. Thus, the recording unit can analyze voice and record it as text data. Specifically, the recording unit performs feature extraction processing such as spectrogram conversion and Mel-frequency cepstral coefficient (MFCC) extraction on the preprocessed voice data received from the reception unit. The recording unit inputs the obtained feature vectors (e.g., 40-dimensional MFCC×100 frames) into a convolutional neural network (CNN), recurrent neural network (RNN), or Transformer-based speech recognition model. These models use pre-trained weights to generate string sequences (e.g., “I was diagnosed with gastritis at the hospital”) from voice with high accuracy and speed. Furthermore, the natural language processing module performs morphological analysis and named entity recognition (NER) on the obtained text data, and converts it into structured data such as “diagnosis name,” “hospital name,” and “date” (e.g., diagnosis: gastritis, hospital: XX, date: 2024-05-01). The recording unit stores these structured data in a database in chronological order, greatly improving the searchability and reusability of the data. Examples of input to the AI include feature vectors extracted from voice and past history data (e.g., “diagnosed with gastritis on 2024-05-01”), and examples of AI output include text data (e.g., “I was diagnosed with gastritis at the hospital”) and structured data (e.g., diagnosis: gastritis, hospital: XX, date: 2024-05-01). These outputs are used by subsequent display units, reminder units, suggestion units, for display on the user interface, and as input to health advice generation modules. As a technical effect, the recording unit automatically generates highly accurate text and structured data from voice input, greatly improving recording accuracy, searchability, and reusability compared to conventional manual input or paper records. Furthermore, since it efficiently generates data that serves as the basis for history analysis by AI and individually optimized reminders and suggestions, the technical value of the entire health management system is enhanced. Application fields include personal health management, telemedicine, automation of records in nursing care settings, and corporate health management support.

The display unit is capable of displaying the data recorded by the recording unit in chronological order. For example, the display unit displays the data recorded by the recording unit in chronological order. The display unit displays data based on the format of timestamps and display order. For example, the display unit displays a list of which hospital provided which diagnosis in the past, which medication was taken and when, which examination was received and when, and what the results were. Thus, the display unit can display recorded data in chronological order. Specifically, the display unit extracts structured data (e.g., diagnosis name, hospital name, date, medication name, examination name, examination result, etc.) received from the recording unit from the database, sorts it by timestamp and category, and displays it as a list or graph in chronological order. The display unit provides multiple display modes on the user interface, such as calendar format, timeline format, and graph format (e.g., transition graph of examination values), allowing the user to intuitively grasp the history. Furthermore, the display unit can automatically emphasize or color-code the display according to the importance and category of the data (e.g., medical-related, daily life-related, highly urgent data, etc.). Examples of input to the AI include structured data stored in chronological order (e.g., “2024-05-01 gastritis diagnosis,” “2024-04-10 blood test”), and examples of AI output include chronological list display, graph display, and highlight display of important events. These outputs are used for the user to quickly and accurately grasp past health status and treatment history. As a technical effect, the display unit automatically organizes and visualizes large amounts of health data in chronological order, greatly improving the efficiency and accuracy of information grasping compared to conventional paper records or manual searches. Furthermore, by linking with AI data analysis results and reminder/suggestion information, it promotes user health management behavior and reduces the risk of missed examinations and delayed treatment. Application fields include personal health management, telemedicine, automation of records in nursing care settings, and corporate health management support.

The reminder unit is capable of providing reminders when periodic examinations are required. For example, the reminder unit provides reminders when periodic examinations are required. The reminder unit provides reminders based on the format of notifications and the timing of reminders. For example, by providing a reminder such as “The next blood test is next month,” the reminder unit helps the user not forget the examination. Thus, the reminder unit can provide reminders when periodic examinations are required. Specifically, the reminder unit scans examination history data obtained from the recording unit, determines the recommended interval for examinations (e.g., blood tests every 6 months, gastroscopy every 2 years) using rule-based or machine learning models (e.g., decision trees, random forests), and calculates the next scheduled examination date. The reminder unit also considers the user's calendar information and past examination tendencies (e.g., tendency to delay examinations, preferred days of the week for visits) to determine the optimal notification timing (e.g., one week before the scheduled examination date, the day before, etc.). Notification formats may include push notifications, email, voice announcements, calendar integration, and other means that can be automatically selected. Examples of input to the AI include examination history data (e.g., “2024-04-10 blood test”), user calendar information, and past reminder history, and examples of AI output include “next blood test recommended on 2024-10-10,” “examination reminder notification (2024-10-03),” etc. These outputs are used for notifications to the user and as input to subsequent health advice generation modules. As a technical effect, the reminder unit automatically generates optimal reminders based on examination history and user attributes, greatly reducing the risk of missed examinations and delayed treatment compared to conventional manual management or calendar entries. Furthermore, individually optimized reminders by AI promote user health management behavior and contribute to health maintenance and disease prevention. Application fields include personal health management, telemedicine, automation of records in nursing care settings, and corporate health management support.

The suggestion unit is capable of indicating body parts that have not been examined recently and proposing appropriate human health checkup menus and nearby hospitals. For example, the suggestion unit indicates body parts that have not been examined recently and proposes appropriate human health checkup menus and nearby hospitals. The suggestion unit determines body parts that have not been examined recently based on past examination history and recommended examination frequency. For example, the suggestion unit may propose, “You have not had a stomach examination recently, so we recommend a gastroscopy. You can receive it at a nearby hospital.” Thus, the suggestion unit can indicate body parts that have not been examined recently and propose appropriate human health checkup menus and nearby hospitals. Specifically, the suggestion unit performs gap analysis of examination history data obtained from the recording unit (e.g., gastroscopy not performed in the past two years) and uses user attributes (age, gender, medical history, etc.) as input to generate suggestion sentences such as “gastroscopy recommended” using rule-based or large language models. Furthermore, the suggestion unit links with a geographic information database to search for a list of nearby medical institutions based on the user's current location and incorporates this into the suggestion sentence. Examples of input to the AI include examination history data (e.g., “gastroscopy not performed on 2022-05-01”), user attributes (e.g., age 50, male, medical history: hypertension), and current location information (e.g., Chiyoda-ku, Tokyo), and examples of AI output include “gastroscopy recommended,” “examination available at the nearest XX hospital,” etc. These outputs are used for display and notification in the user interface, as well as input to subsequent health advice generation modules. As a technical effect, the suggestion unit automatically generates individually optimized examination suggestions and medical institution guidance based on examination history, user attributes, and geographic information, greatly improving suggestion accuracy and convenience compared to conventional uniform health checkup guidance. Furthermore, natural language generation by AI and linkage with geographic information promote user health management behavior and reduce the risk of missed examinations and delayed treatment. Application fields include personal health management, telemedicine, automation of records in nursing care settings, and corporate health management support.

The reception unit is capable of estimating the user's emotion and adjusting the timing of receiving voice input based on the estimated emotion. For example, the reception unit estimates the user's emotion and adjusts the timing of receiving voice input based on the estimated emotion. Emotion estimation is realized using an emotion engine or generative AI, for example, by employing an emotion estimation function. The generative AI may be a text generation AI (e.g., LLM) or a multimodal generative AI, but is not limited to these examples. For instance, if the user is feeling stressed, the timing of receiving voice input is delayed to provide a relaxing environment. If the user is relaxed, the timing of receiving voice input is advanced to smoothly receive input. Furthermore, if the user is in a hurry, the timing of receiving voice input is set to immediate to quickly receive input. Thus, the timing of receiving voice input can be adjusted based on the user's emotion. Specifically, the reception unit acquires the user's voice input data (e.g., one-dimensional chronological voice data sampled at 16 kHz, length 80,000 samples), performs preprocessing such as noise reduction using spectral subtraction or Wiener filter and volume normalization, and then extracts voice features (e.g., 40-dimensional MFCC×100 frames). The reception unit inputs these feature vectors into a convolutional neural network (CNN), recurrent neural network (RNN), or Transformer-based speech emotion recognition model to perform emotion classification. Examples of input to the AI include emotion-labeled voice data such as “anger,” “sadness,” “relaxation,” “stress,” “tension,” and actual user utterances (e.g., “I am very tired today,” “I am in a hurry”). The reception unit obtains output from the model such as emotion categories (e.g., stress=0.85, relaxation=0.10, tension=0.05 probability distribution) or emotion intensity scores (e.g., stress level 0.7, relaxation level 0.2). These output values are input to the subsequent reception timing control module, and, for example, if the stress level is high, the reception timing is delayed; if the relaxation level is high, immediate reception; if the urgency level is high, prioritized reception, and so on, are automatically executed. Furthermore, the reception unit accumulates the user's past emotion estimation history and reception timing optimization parameters, and may perform personalization of reception timing control using reinforcement learning algorithms or Bayesian optimization. As a technical effect, the reception unit realizes automatic emotion estimation in a high-dimensional feature space of voice signals and real-time reception timing control, which is difficult to achieve with mere human emotion observation or manual reception, thereby reducing the user's psychological burden and optimizing speech recognition accuracy and system responsiveness. Compared to conventional uniform reception methods, the quality of user experience, recording accuracy, and overall system processing efficiency are greatly improved. Application fields include not only health management systems, but also telemedicine, automation of records in nursing care settings, stress management support, and emotion-adaptive user interfaces.

The reception unit is capable of analyzing the user's past voice input history and selecting an optimal reception method. For example, the reception unit analyzes the user's past voice input history and selects an optimal reception method. The generative AI analyzes the past voice input history and selects an optimal reception method. For instance, the reception unit may preferentially propose the voice input method most frequently used by the user in the past. Additionally, based on the user's past voice input history, the reception unit may propose the optimal reception method for specific time periods. Furthermore, the reception unit may analyze the user's past voice input history and select the most efficient reception method. Thus, the reception unit can analyze the user's past voice input history and select an optimal reception method. Specifically, the reception unit extracts voice input history data accumulated in chronological order for each user (e.g., structured data including input date and time, input device type, input environment, input content length, and success rate) from the database. The reception unit applies history analysis algorithms using decision trees, random forests, or large language models to these history data as input, and automatically extracts features (e.g., input frequency, usage trends by time period, recognition accuracy by device, success rate under noisy environments, etc.). Examples of input to the AI include history records such as “2024-05-01 08:00 smartphone voice input success,” “2024-05-02 20:00 wearable microphone input failure.” Examples of AI output include reception method proposals such as “smartphone voice input recommended in the morning,” “noise-canceling headset recommended at night,” “use text input in noisy environments,” and reception method priority lists (e.g., smartphone>wearable microphone>in-vehicle microphone). The reception unit automatically selects the optimal reception method based on these outputs by matching with the user's current usage status and environmental information, and proposes or switches methods on the user interface. Furthermore, the selection logic for reception methods is sequentially learned and optimized according to each user's history patterns and changes in reception success rate, so usability, recognition accuracy, and reception efficiency are greatly improved compared to conventional uniform reception methods. As a technical effect, the reception unit analyzes large amounts of history data in a high-dimensional feature space, realizes personalization and automatic optimization of reception methods, and flexibly responds to diverse usage environments and needs for each user. Application fields include health management systems, telemedicine, automation of records in nursing care settings, corporate health management support, and voice assistant systems.

The reception unit is capable of performing filtering based on the user's current health status and areas of interest when receiving voice input. For example, the reception unit performs filtering based on the user's current health status and areas of interest when receiving voice input. The generative AI filters voice input based on the user's current health status and areas of interest. For instance, the reception unit may receive only relevant voice input based on the user's current health status. Additionally, the reception unit may preferentially receive relevant voice input based on the user's areas of interest. Furthermore, the reception unit may filter out unnecessary voice input based on the user's health status and areas of interest. Thus, the reception unit can filter voice input based on the user's current health status and areas of interest. Specifically, the reception unit acquires the user's health status data (e.g., recent diagnosis name, medication history, examination results, stress level, etc.) and areas of interest data (e.g., categories frequently recorded by the user in the past, interest tags set by the user, etc.) from the database. When the reception unit receives voice input data (e.g., 16 kHz sampled voice waveform), it first converts it to text using a speech recognition model (CNN, RNN, Transformer, etc.), and then analyzes the content using a natural language processing module (e.g., morphological analysis, topic classification, named entity recognition). Examples of input to the AI include health status data such as “2024-05-01 gastritis diagnosis,” “2024-05-02 lack of exercise,” “2024-05-03 sleep disorder,” and interest tags such as “diet,” “exercise,” “sleep.” Examples of AI output include filtering decisions and priority lists such as “today's voice input is related to health status and accepted,” “input outside areas of interest is put on hold,” “sleep-related input is accepted with priority.” The reception unit automatically determines acceptance or priority based on these outputs, does not accept unnecessary input, and sends only highly relevant input to subsequent processing. Furthermore, the filtering algorithm is sequentially learned and optimized according to changes in the user's health status and areas of interest, so recording accuracy, usability, and system efficiency are greatly improved compared to conventional uniform reception methods. As a technical effect, the reception unit realizes input filtering adapted to each user's health status and areas of interest, prevents unnecessary data accumulation and erroneous records, and improves the accuracy of important information recording. Application fields include health management systems, telemedicine, automation of records in nursing care settings, and personalized healthcare services.

The reception unit is capable of estimating the user's emotion and determining the priority of voice input to be received based on the estimated emotion. For example, the reception unit estimates the user's emotion and determines the priority of voice input to be received based on the estimated emotion. Emotion estimation is realized using an emotion engine or generative AI, for example, by employing an emotion estimation function. The generative AI may be a text generation AI (e.g., LLM) or a multimodal generative AI, but is not limited to these examples. For instance, if the user is feeling stressed, important voice input is accepted with priority. If the user is relaxed, all voice input is accepted equally. Furthermore, if the user is in a hurry, urgent voice input is accepted with priority. Thus, the reception unit can determine the priority of voice input to be received based on the user's emotion. Specifically, the reception unit acquires the user's voice input data (e.g., 16 kHz sampled voice waveform), performs preprocessing and feature extraction (e.g., MFCC, spectrogram), and then estimates emotion using a speech emotion recognition model (CNN, RNN, Transformer, etc.). The reception unit outputs estimated emotion categories (e.g., stress, relaxation, tension, anger) and emotion intensity scores (e.g., stress level 0.8), and combines these with analysis results of the voice input content (e.g., diagnosis name, urgency, category) to apply a priority determination algorithm (e.g., weighted scoring, rule-based, machine learning model). Examples of input to the AI include “voice input A: stress level 0.9, content: urgent diagnosis,” “voice input B: relaxation level 0.7, content: daily record.” Examples of AI output include priority lists and reception order such as “accept voice input A with highest priority,” “accept voice input B as normal.” The reception unit automatically adjusts the order of receiving voice input based on these outputs, and sends highly important or urgent input to subsequent processing with priority. Furthermore, the priority determination logic is sequentially learned and optimized based on the user's emotion estimation history and feedback from reception results, so emergency response capability, recording accuracy, and user satisfaction are greatly improved compared to conventional uniform reception methods. As a technical effect, the reception unit realizes priority control by combining emotion estimation and content analysis, enabling flexible reception processing according to the user's psychological state and situation. Application fields include health management systems, telemedicine, automation of records in nursing care settings, and emergency notification systems.

The reception unit is capable of prioritizing highly relevant information by considering the user's geographic location when receiving voice input. For example, the reception unit prioritizes highly relevant information by considering the user's geographic location when receiving voice input. The generative AI receives voice input by considering the user's geographic location. For instance, if the user is in a specific region, the reception unit prioritizes voice input related to that region. Additionally, if the user is traveling, the reception unit prioritizes voice input related to the travel destination. Furthermore, if the user is at home, the reception unit prioritizes voice input related to the home. Thus, the reception unit can receive voice input by considering the user's geographic location. Specifically, the reception unit acquires geographic location data (e.g., latitude/longitude, address, facility name) obtained in real time from the user's device via GPS, Wi-Fi positioning, or base station information, and links it with voice input data for reception processing. The reception unit converts the voice input content to text using a speech recognition model, and analyzes the content using a natural language processing module (e.g., place name extraction, facility name extraction, category classification). Examples of input to the AI include “current location: Chiyoda-ku, Tokyo, voice input: request for gastroscopy,” “current location: Kita-ku, Osaka, voice input: request for examination at a nearby hospital.” Examples of AI output include priority lists and acceptance decisions such as “prioritize acceptance of medical institution-related input within Chiyoda-ku,” “prioritize acceptance of health consultation input at travel destination,” “accept health record input related to home as normal.” The reception unit sends highly geographically relevant voice input to subsequent processing with priority based on these outputs, and holds or accepts less relevant input with lower priority. Furthermore, by linking with a geographic information database and dynamically acquiring and utilizing medical institution information and health service information for each region, the accuracy and convenience of reception processing are improved. As a technical effect, the reception unit realizes reception optimization utilizing the user's geographic location information, contributing to improved adaptation to regional health issues and access to medical resources. Compared to conventional uniform reception methods, regional adaptability, user satisfaction, and system efficiency are greatly improved. Application fields include health management systems, telemedicine, regional healthcare collaboration, and health support services for travelers.

The reception unit is capable of analyzing the user's social media activity when receiving voice input and receiving relevant information. For example, the reception unit analyzes the user's social media activity when receiving voice input and receives relevant information. The generative AI analyzes the user's social media activity and receives relevant information. For instance, the reception unit may prioritize voice input related to topics of interest extracted from the user's social media activity. Additionally, the reception unit may analyze the user's social media activity and filter relevant information. Furthermore, the reception unit may filter out unnecessary voice input based on the user's social media activity. Thus, the reception unit can analyze the user's social media activity and receive relevant information. Specifically, the reception unit periodically collects and accumulates structured data such as post history, comments, like history, and follow relationships (e.g., post date/time, topic tags, emotion labels, post content text) obtained via API from multiple social media platforms used by the user (e.g., microblogs, photo sharing services, health community sites). When receiving voice input, the reception unit applies natural language processing algorithms (e.g., BERT-based topic classification model, emotion analysis model, clustering algorithm) to these social media activity data as input, and automatically extracts the user's interest topic distribution (e.g., interest scores for categories such as health, exercise, diet, sleep, stress) and recent topic trends. Examples of input to the AI include history data such as “2024-05-01 # exercise post,” “2024-05-02 # sleep comment,” “2024-05-03 # stress like,” and post text (e.g., “I recently started jogging,” “I have been sleep deprived”). When the reception unit receives voice input data (e.g., 16 kHz sampled voice waveform), it converts it to text using a speech recognition model (CNN, RNN, Transformer, etc.), analyzes the content (e.g., topic classification, keyword extraction), and matches it with interest topics extracted from social media activity. Examples of AI output include filtering decisions and priority lists such as “today's voice input matches # exercise topic and is accepted with priority,” “# diet-related input is put on hold due to low interest,” “# stress-related input is accepted as highly important.” The reception unit automatically determines acceptance or priority based on these outputs, and sends only highly relevant input to subsequent recording units or health advice generation modules. Furthermore, the filtering algorithm is sequentially learned and optimized according to changes in the user's social media activity and the emergence of new interest topics, so recording accuracy, usability, and system efficiency are greatly improved compared to conventional uniform reception methods. As a technical effect, the reception unit realizes input filtering adapted to each user's interest topics and behavioral tendencies, prevents unnecessary data accumulation and erroneous records, and improves the accuracy of important information recording. Application fields include health management systems, personalized healthcare services, telemedicine, automation of records in nursing care settings, corporate health management support, as well as user life log analysis and well-being support services.

The recording unit is capable of estimating the user's emotion and adjusting the method of expression in the record based on the estimated emotion. For example, the recording unit estimates the user's emotion and adjusts the method of expression in the record based on the estimated emotion. Emotion estimation is realized using an emotion engine or generative AI, for example, by employing an emotion estimation function. The generative AI may be a text generation AI (e.g., LLM) or a multimodal generative AI, but is not limited to these examples. For instance, if the user is relaxed, detailed records are made. If the user is in a hurry, concise records are made. Furthermore, if the user is feeling stressed, only important information is recorded. Thus, the recording unit can adjust the method of expression in the record based on the user's emotion. Specifically, the recording unit performs feature extraction processing such as spectrogram conversion and MFCC extraction on the voice input data received from the reception unit (e.g., 16 kHz sampled voice waveform), and estimates emotion categories (e.g., relaxation, stress, tension, anger) and emotion intensity scores (e.g., stress level 0.8, relaxation level 0.1) using a speech emotion recognition model (CNN, RNN, Transformer, etc.). The recording unit combines the estimated emotion information with the text data obtained from the speech recognition model, and inputs it to a natural language generation model (large language model, etc.) to automatically select the method of expression in the record (e.g., detailed record, concise record, key point extraction record, etc.). Examples of input to the AI include “voice input: I am very tired today, emotion estimation: stress level 0.9,” “voice input: I feel good today, emotion estimation: relaxation level 0.8.” Examples of AI output include “detailed record: Today I felt strong fatigue from work and felt the need for rest,” “concise record: tired,” “key point record: only important matters recorded.” The recording unit automatically adjusts the granularity and expression style of the record data (e.g., detailed explanation, bullet points, summary format) based on these outputs, and stores the optimal record in the database according to the user's psychological state and situation. Furthermore, the selection logic for the method of expression in the record is sequentially learned and optimized based on the user's emotion estimation history and feedback from record usage, so recording accuracy, usability, and information searchability are greatly improved compared to conventional uniform recording methods. As a technical effect, the recording unit realizes optimization of record expression by combining emotion estimation and natural language generation, enabling flexible record management according to the user's psychological state and usage scene. Application fields include health management systems, telemedicine, automation of records in nursing care settings, personalized healthcare services, and stress management support.

The recording unit is capable of adjusting the level of detail in the record based on the importance of the voice input at the time of recording. For example, the recording unit adjusts the level of detail in the record based on the importance of the voice input at the time of recording. The generative AI adjusts the level of detail in the record based on the importance of the voice input. For instance, for important voice input, detailed records are made. For general voice input, concise records are made. Furthermore, for urgent voice input, records are made promptly. Thus, the recording unit can adjust the level of detail in the record based on the importance of the voice input. Specifically, the recording unit converts voice input data to text using a speech recognition model, applies a natural language processing module (e.g., named entity recognition, topic classification, urgency determination algorithm, etc.), and automatically determines the importance score (e.g., 0.0 to 1.0) and urgency label (e.g., high, medium, low) of the input content. The recording unit inputs this importance information to a natural language generation model (large language model, etc.) to automatically select the level of detail in the record (e.g., detailed record, concise record, key point record, immediate record, etc.). Examples of input to the AI include “voice input: persistent chest pain, importance: 0.95, urgency: high,” “voice input: I took a walk today, importance: 0.3, urgency: low.” Examples of AI output include “detailed record: detailed record of symptom progression, onset time, pain level, etc. ,” “concise record: only record of taking a walk,” “immediate record: immediate record due to high urgency.” The recording unit automatically adjusts the granularity and timing of record data based on these outputs, recording important information in detail, general information concisely, and urgent information immediately. Furthermore, the selection logic for the level of detail in the record is sequentially learned and optimized based on the user's usage history and feedback from record reference frequency, so recording accuracy, information searchability, and system responsiveness are greatly improved compared to conventional uniform recording methods. As a technical effect, the recording unit realizes optimization of record detail according to the importance and urgency of voice input, supporting user health management behavior and rapid decision-making in medical settings. Application fields include health management systems, telemedicine, automation of records in nursing care settings, emergency notification systems, and personalized healthcare services.

The recording unit is capable of applying different recording algorithms according to the category of the voice input at the time of recording. For example, the recording unit applies different recording algorithms according to the category of the voice input at the time of recording. The generative AI applies different recording algorithms according to the category of the voice input. For instance, for medical-related voice input, a detailed recording algorithm is applied. For voice input related to daily life, a concise recording algorithm is applied. Furthermore, for urgent voice input, a rapid recording algorithm is applied. Thus, the recording unit can apply different recording algorithms according to the category of the voice input. Specifically, the recording unit converts voice input data to text using a speech recognition model, applies a natural language processing module (e.g., topic classification model, category classification algorithm, etc.), and automatically determines the category of the input content (e.g., medical, daily life, exercise, diet, sleep, emergency, etc.). The recording unit inputs the determined category information to a category-optimized recording algorithm (e.g., detailed chronological record and symptom progression record for medical category, key point extraction and concise record for daily life category, immediate record and alert generation for emergency category), and generates and stores the record data. Examples of input to the AI include “voice input: I was diagnosed with gastritis at the hospital, category: medical,” “voice input: I jogged for 30 minutes today, category: exercise,” “voice input: I have chest pain, category: emergency.” Examples of AI output include “medical category: detailed record of diagnosis name, hospital name, date, symptom progression,” “exercise category: concise record of exercise type, time, calories burned,” “emergency category: immediate record and alert notification.” The recording unit automatically adjusts the granularity, record items, and timing of record data for each category based on these outputs, realizing optimal record management according to the user's purpose and situation. Furthermore, the selection logic for recording algorithms is sequentially learned and optimized based on the user's usage history and feedback from record reference frequency, so recording accuracy, information searchability, and system efficiency are greatly improved compared to conventional uniform recording methods. As a technical effect, the recording unit realizes optimization of recording algorithms according to the category of voice input, supporting user health management behavior and efficiency of recording operations in medical settings. Application fields include health management systems, telemedicine, automation of records in nursing care settings, personalized healthcare services, and emergency notification systems.

The recording unit is capable of estimating the user's emotion and adjusting the length of the record based on the estimated emotion. For example, the recording unit estimates the user's emotion and adjusts the length of the record based on the estimated emotion. Emotion estimation is realized using an emotion engine or generative AI, for example, by employing an emotion estimation function. The generative AI may be a text generation AI (e.g., LLM) or a multimodal generative AI, but is not limited to these examples. For instance, if the user is relaxed, detailed records are made. If the user is in a hurry, concise records are made. Furthermore, if the user is feeling stressed, only important information is recorded. Thus, the recording unit can adjust the length of the record based on the user's emotion. Specifically, the recording unit estimates emotion categories and emotion intensity scores using a speech emotion recognition model (CNN, RNN, Transformer, etc.) on the voice input data received from the reception unit, and combines it with the content converted to text by the speech recognition model, inputting it to a natural language generation model (large language model, etc.). The recording unit automatically selects the length of the record (e.g., detailed record, concise record, key point record, etc.) according to the emotion information, and generates and stores the record data. Examples of input to the AI include “voice input: I am very tired today, emotion estimation: stress level 0.9,” “voice input: I feel good today, emotion estimation: relaxation level 0.8.” Examples of AI output include “detailed record: Today I felt strong fatigue from work and felt the need for rest,” “concise record: tired,” “key point record: only important matters recorded.” The recording unit automatically adjusts the length and expression style of the record data based on these outputs, and stores the optimal record in the database according to the user's psychological state and situation. Furthermore, the selection logic for record length is sequentially learned and optimized based on the user's emotion estimation history and feedback from record usage, so recording accuracy, usability, and information searchability are greatly improved compared to conventional uniform recording methods. As a technical effect, the recording unit realizes optimization of record length by combining emotion estimation and natural language generation, enabling flexible record management according to the user's psychological state and usage scene. Application fields include health management systems, telemedicine, automation of records in nursing care settings, personalized healthcare services, and stress management support.

The recording unit is capable of determining the priority of the record based on the submission timing of the voice input at the time of recording. For example, the recording unit determines the priority of the record based on the submission timing of the voice input at the time of recording. The generative AI determines the priority of the record based on the submission timing of the voice input. For instance, for urgent voice input, records are made with priority. For general voice input, records are made with normal priority. Furthermore, for voice input submitted late, records are made later. Thus, the recording unit can determine the priority of the record based on the submission timing of the voice input. Specifically, the recording unit acquires timestamp information (e.g., input date/time, reception time, submission delay time) attached to the voice input data, and applies a natural language processing module (e.g., urgency determination algorithm, submission delay determination model, etc.) together with the content converted to text by the speech recognition model. The recording unit automatically determines the priority (e.g., high, medium, low) and order of the record based on the submission timing and urgency, and controls the recording so that high-priority input is recorded immediately, normal input is recorded sequentially, and delayed input is recorded later. Examples of input to the AI include “voice input: persistent chest pain, submission time: 2024-05-01 08:00, urgency: high,” “voice input: I took a walk today, submission time: 2024-05-01 20:00, urgency: low,” “voice input: yesterday's event, submission delay: 24 hours.” Examples of AI output include “priority record: record chest pain input immediately,” “normal record: record walk input in normal order,” “delayed record: record delayed input later.” The recording unit automatically adjusts the order and timing of record data based on these outputs, supporting user health management behavior and rapid decision-making in medical settings. Furthermore, the decision logic for record priority is sequentially learned and optimized based on the user's usage history and feedback from record reference frequency, so recording accuracy, information searchability, and system responsiveness are greatly improved compared to conventional uniform recording methods. As a technical effect, the recording unit realizes optimization of record priority according to the submission timing and urgency of voice input, supporting user health management behavior and efficiency of recording operations in medical settings. Application fields include health management systems, telemedicine, automation of records in nursing care settings, emergency notification systems, and personalized healthcare services.

The recording unit is capable of adjusting the order of the record based on the relevance of the voice input at the time of recording. For example, the recording unit adjusts the order of the record based on the relevance of the voice input at the time of recording. The generative AI adjusts the order of the record based on the relevance of the voice input. For instance, for important voice input, records are made with priority. For general voice input, records are made in the normal order. Furthermore, for voice input with low relevance, records are made later. Thus, the recording unit can adjust the order of the record based on the relevance of the voice input. Specifically, the recording unit converts voice input data to text using a speech recognition model, applies a natural language processing module (e.g., topic classification, relevance scoring algorithm, etc.), and automatically calculates the relevance score (e.g., 0.0 to 1.0) between the input content and the user's health status/history data. The recording unit inputs the relevance score and importance information to a record order determination algorithm (e.g., weighted scoring, rule-based, machine learning model, etc.), and controls the recording so that highly relevant input is recorded with priority, general input is recorded in the normal order, and low-relevance input is recorded later. Examples of input to the AI include “voice input: gastritis diagnosis, relevance score: 0.95,” “voice input: weather talk, relevance score: 0.1,” “voice input: exercise record, relevance score: 0.7.” Examples of AI output include “priority record: record gastritis diagnosis input with highest priority,” “normal record: record exercise record in normal order,” “delayed record: record weather talk later.” The recording unit automatically adjusts the order and timing of record data based on these outputs, supporting user health management behavior and efficiency of recording operations in medical settings. Furthermore, the decision logic for record order is sequentially learned and optimized based on the user's usage history and feedback from record reference frequency, so recording accuracy, information searchability, and system efficiency are greatly improved compared to conventional uniform recording methods. As a technical effect, the recording unit realizes optimization of record order according to the relevance of voice input, supporting user health management behavior and efficiency of recording operations in medical settings. Application fields include health management systems, telemedicine, automation of records in nursing care settings, and personalized healthcare services.

The display unit can estimate the user's emotion and adjust the display method based on the estimated emotion. For example, the display unit estimates the user's emotion and adjusts the display method according to the estimated emotion. Emotion estimation is realized, for example, by using an emotion estimation function with an emotion engine or generative AI. Generative AI may include text generation AI (e.g., LLM) or multimodal generative AI, but is not limited thereto. For example, when the user is relaxed, detailed information is displayed. When the user is in a hurry, concise information is displayed. Furthermore, when the user is feeling stressed, only important information is displayed. Thus, the display method can be adjusted based on the user's emotion. Specifically, the display unit receives voice input data (e.g., one-dimensional time-series voice waveform sampled at 16 kHz, length 80,000 samples) from the reception unit or recording unit, performs feature extraction processing such as spectrogram conversion or MFCC extraction, and estimates emotion categories (e.g., relaxed, stressed, tense, angry, etc.) and emotion intensity scores (e.g., stress level 0.8, relaxation level 0.1) using a voice emotion recognition model (convolutional neural network, recurrent neural network, Transformer-based model, etc.). The display unit combines the estimated emotion information with structured data obtained from the recording unit (e.g., diagnosis name, test results, medication name, date, etc.), and inputs them into a natural language generation model (large language model, etc.) or rule-based display control algorithm to automatically select the display method (e.g., detailed display, concise display, key point emphasis display, etc.). Examples of AI input include: “Voice input: I am very tired today, emotion estimation: stress level 0.9”; “Voice input: I feel good today, emotion estimation: relaxation level 0.8”. Examples of AI output include: “Detailed display: display diagnosis details, test results, and medication history in chronological order in detail”; “Concise display: display only today's important items in bullet points”; “Key point emphasis display: emphasize only highly urgent information”. Based on these outputs, the display unit automatically controls selection of display items, adjustment of display granularity, color coding and emphasis, and optimization of display order on the user interface. Furthermore, the logic for selecting the display method is sequentially learned and optimized based on the user's emotion estimation history and feedback from display usage (e.g., tendency to prefer detailed display, tendency to view only key points when stressed), resulting in greatly improved information comprehension efficiency, usability, and satisfaction compared to conventional uniform display methods. As a technical effect, the display unit realizes personalized display optimization by combining emotion estimation and display control, enabling flexible information presentation according to the user's psychological state and usage scene. Application fields include health management systems, telemedicine, automated record-keeping in nursing care settings, personalized healthcare services, stress management support, and emotion-adaptive user interfaces.

The display unit can adjust the level of detail in the display based on the importance of the recorded data at the time of display. For example, the display unit adjusts the level of detail in the display according to the importance of the recorded data at the time of display. Generative AI adjusts the level of detail in the display based on the importance of the recorded data. For example, for important data, detailed information is displayed. For general data, concise information is displayed. Furthermore, for highly urgent data, the display is performed promptly. Thus, the level of detail in the display can be adjusted based on the importance of the recorded data. Specifically, the display unit applies a natural language processing module (named entity extraction, topic classification, urgency determination algorithm, etc.) to structured data obtained from the recording unit (e.g., diagnosis name, test results, medication name, date, urgency label, etc.), and automatically determines the importance score (e.g., 0.0 to 1.0) and urgency label (e.g., high, medium, low) for each data item. The display unit uses this importance information as input to a natural language generation model or rule-based display control algorithm to automatically select the display detail level (e.g., detailed display, concise display, key point display, immediate display, etc.). Examples of AI input include: “Data: persistent chest pain, importance: 0.95, urgency: high”; “Data: went for a walk today, importance: 0.3, urgency: low”. Examples of AI output include: “Detailed display: show the course of symptoms, onset time, pain level, etc. in detail”; “Concise display: show only the fact of walking”; “Immediate display: display immediately due to high urgency”. Based on these outputs, the display unit automatically adjusts the granularity and timing of display items on the user interface, displaying important information in detail, general information concisely, and highly urgent information immediately. Furthermore, the logic for selecting the display detail level is sequentially learned and optimized based on user usage history and feedback on display reference frequency, resulting in greatly improved information comprehension efficiency, accuracy, and system responsiveness compared to conventional uniform display methods. As a technical effect, the display unit realizes optimization of display detail level according to the importance and urgency of data, supporting health management actions and rapid decision-making in medical settings. Application fields include health management systems, telemedicine, automated record-keeping in nursing care settings, emergency notification systems, and personalized healthcare services.

The display unit can apply different display algorithms according to the category of the data at the time of display. For example, the display unit applies different display algorithms according to the category of the data at the time of display. Generative AI applies different display algorithms according to the category of the data. For example, for medical-related data, a detailed display algorithm is applied. For data related to daily life, a concise display algorithm is applied. Furthermore, for highly urgent data, a rapid display algorithm is applied. Thus, different display algorithms can be applied according to the category of the data. Specifically, the display unit applies a natural language processing module (topic classification model, category classification algorithm, etc.) to structured data obtained from the recording unit (e.g., diagnosis name, test results, medication name, date, category label, etc.), and automatically determines the category of the input content (e.g., medical, daily life, exercise, diet, sleep, emergency, etc.). The display unit uses the determined category information as input to automatically select the optimal display algorithm for each category (e.g., for medical category: detailed chronological display and symptom progression graph; for daily life category: key point extraction and concise display; for emergency category: immediate display and alert emphasis, etc.), and generates and presents the display data. Examples of AI input include: “Data: diagnosed with gastritis at hospital, category: medical”; “Data: jogged for 30 minutes today, category: exercise”; “Data: chest pain, category: emergency”. Examples of AI output include: “Medical category: display diagnosis name, hospital name, date, and symptom progression in detail”; “Exercise category: display exercise type, duration, and calories burned concisely”; “Emergency category: immediate display and alert notification”. Based on these outputs, the display unit automatically adjusts the granularity, items, and timing of display for each category, realizing optimal information presentation according to the user's purpose and situation. Furthermore, the logic for selecting the display algorithm is sequentially learned and optimized based on user usage history and feedback on display reference frequency, resulting in greatly improved information comprehension efficiency, accuracy, and system efficiency compared to conventional uniform display methods. As a technical effect, the display unit realizes optimization of display algorithms according to the category of data, supporting health management actions and efficient information presentation in medical settings. Application fields include health management systems, telemedicine, automated record-keeping in nursing care settings, personalized healthcare services, and emergency notification systems.

The display unit can estimate the user's emotion and adjust the length of the display based on the estimated emotion. For example, the display unit estimates the user's emotion and adjusts the length of the display according to the estimated emotion. Emotion estimation is realized, for example, by using an emotion estimation function with an emotion engine or generative AI. Generative AI may include text generation AI (e.g., LLM) or multimodal generative AI, but is not limited thereto. For example, when the user is relaxed, detailed information is displayed. When the user is in a hurry, concise information is displayed. Furthermore, when the user is feeling stressed, only important information is displayed. Thus, the length of the display can be adjusted based on the user's emotion. Specifically, the display unit receives voice input data from the reception unit or recording unit, estimates emotion categories and emotion intensity scores using a voice emotion recognition model (convolutional neural network, recurrent neural network, Transformer, etc.), combines them with structured data obtained from the recording unit, and inputs them into a natural language generation model (large language model, etc.) or rule-based display control algorithm. The display unit automatically selects the length of the display (e.g., detailed display, concise display, key point display, etc.) according to the emotion information, and generates and presents the display data. Examples of AI input include: “Voice input: I am very tired today, emotion estimation: stress level 0.9”; “Voice input: I feel good today, emotion estimation: relaxation level 0.8”. Examples of AI output include: “Detailed display: I felt very fatigued at work today and need rest”; “Concise display: tired”; “Key point display: display only important items”. Based on these outputs, the display unit automatically adjusts the length and expression style of the display data, realizing optimal information presentation on the user interface according to the user's psychological state and situation. Furthermore, the logic for selecting the display length is sequentially learned and optimized based on the user's emotion estimation history and feedback from display usage, resulting in greatly improved information comprehension efficiency, usability, and information searchability compared to conventional uniform display methods. As a technical effect, the display unit realizes optimization of display length by combining emotion estimation and natural language generation, enabling flexible information presentation according to the user's psychological state and usage scene. Application fields include health management systems, telemedicine, automated record-keeping in nursing care settings, personalized healthcare services, and stress management support.

The display unit can determine the priority of display based on the submission timing of the data at the time of display. For example, the display unit determines the priority of display according to the submission timing of the data at the time of display. Generative AI determines the priority of display based on the submission timing of the data. For example, for highly urgent data, display is performed with priority. For general data, display is performed with normal priority. Furthermore, for data with delayed submission, display is performed later. Thus, the priority of display can be determined based on the submission timing of the data. Specifically, the display unit obtains timestamp information attached to data obtained from the recording unit (e.g., input date and time, reception time, submission delay time, etc.), and applies a natural language processing module (urgency determination algorithm, submission delay determination model, etc.). The display unit automatically determines the priority of display (e.g., high, medium, low) and display order based on submission timing and urgency, and controls immediate display for high-priority data, sequential display for normal data, and delayed display for late data. Examples of AI input include: “Data: persistent chest pain, submission time: 2024-05-01 08:00, urgency: high”; “Data: went for a walk today, submission time: 2024-05-01 20:00, urgency: low”; “Data: yesterday's event, submission delay: 24 hours”. Examples of AI output include: “Priority display: immediately display chest pain data”; “Normal display: display walk data in normal order”; “Delayed display: display delayed data later”. Based on these outputs, the display unit automatically adjusts the display order and timing of display data, supporting health management actions and rapid decision-making in medical settings. Furthermore, the logic for determining display priority is sequentially learned and optimized based on user usage history and feedback on display reference frequency, resulting in greatly improved information comprehension efficiency, information searchability, and system responsiveness compared to conventional uniform display methods. As a technical effect, the display unit realizes optimization of display priority according to submission timing and urgency of data, supporting health management actions and efficient information presentation in medical settings. Application fields include health management systems, telemedicine, automated record-keeping in nursing care settings, emergency notification systems, and personalized healthcare services.

The display unit can adjust the display order based on the relevance of the data at the time of display. For example, the display unit adjusts the display order according to the relevance of the data at the time of display. Generative AI adjusts the display order based on the relevance of the data. For example, for important data, display is performed with priority. For general data, display is performed in normal order. Furthermore, for data with low relevance, display is performed later. Thus, the display order can be adjusted based on the relevance of the data. Specifically, the display unit analyzes data obtained from the recording unit using a natural language processing module (topic classification, relevance scoring algorithm, etc.), and automatically calculates the relevance score (e.g., 0.0 to 1.0) between the input content and the user's health status/history data. The display unit uses the relevance score and importance information as input to a display order determination algorithm (weighted scoring, rule-based, machine learning model, etc.), and controls priority display for highly relevant data, normal order display for general data, and delayed display for data with low relevance. Examples of AI input include: “Data: gastritis diagnosis, relevance score: 0.95”; “Data: weather talk, relevance score: 0.1”; “Data: exercise record, relevance score: 0.7”. Examples of AI output include: “Priority display: display gastritis diagnosis data with highest priority”; “Normal display: display exercise record in normal order”; “Delayed display: display weather talk later”. Based on these outputs, the display unit automatically adjusts the display order and timing of display data, supporting health management actions and efficient information presentation in medical settings. Furthermore, the logic for determining display order is sequentially learned and optimized based on user usage history and feedback on display reference frequency, resulting in greatly improved information comprehension efficiency, information searchability, and system efficiency compared to conventional uniform display methods. As a technical effect, the display unit realizes optimization of display order according to the relevance of data, supporting health management actions and efficient information presentation in medical settings. Application fields include health management systems, telemedicine, automated record-keeping in nursing care settings, and personalized healthcare services.

The reminder unit can estimate the user's emotion and adjust the reminder method based on the estimated emotion. For example, the reminder unit estimates the user's emotion and adjusts the reminder method according to the estimated emotion. Emotion estimation is realized, for example, by using an emotion estimation function with an emotion engine or generative AI. Generative AI may include text generation AI (e.g., LLM) or multimodal generative AI, but is not limited thereto. For example, when the user is relaxed, a gentle reminder method is provided. When the user is in a hurry, a rapid reminder method is provided. Furthermore, when the user is feeling stressed, only important reminders are provided. Thus, the reminder method can be adjusted based on the user's emotion. Specifically, the reminder unit receives voice input data (e.g., one-dimensional time-series voice waveform sampled at 16 kHz, length 80,000 samples) from the reception unit or recording unit, performs feature extraction processing such as spectrogram conversion or MFCC extraction, and estimates emotion categories (e.g., relaxed, stressed, tense, angry, etc.) and emotion intensity scores (e.g., stress level 0.8, relaxation level 0.1) using a voice emotion recognition model (convolutional neural network, recurrent neural network, Transformer-based model, etc.). The reminder unit combines the estimated emotion information with the user's health status, history data, and reminder history, and inputs them into a natural language generation model (large language model, etc.) or rule-based reminder control algorithm to automatically select the reminder method (e.g., adjustment of voice tone, softness of notification text, notification frequency, selection of notification means, etc.). Examples of AI input include: “Voice input: I am very tired today, emotion estimation: stress level 0.9”; “Voice input: I feel good today, emotion estimation: relaxation level 0.8”. Examples of AI output include: “Gentle voice notification for reminder”; “Push notification only for highly urgent content”; “Concise text notification”. Based on these outputs, the reminder unit automatically controls selection of notification method, adjustment of notification text, and optimization of notification timing on the user interface. Furthermore, the logic for selecting the reminder method is sequentially learned and optimized based on the user's emotion estimation history and feedback from reminder response history (e.g., reduce notification frequency when stressed, increase detailed notifications when relaxed), resulting in greatly improved acceptance, usability, and behavior promotion effect of reminders compared to conventional uniform notification methods. As a technical effect, the reminder unit realizes personalized notification optimization by combining emotion estimation and reminder control, enabling flexible health management support according to the user's psychological state and usage scene. Application fields include health management systems, telemedicine, automated record-keeping in nursing care settings, personalized healthcare services, stress management support, and emotion-adaptive user interfaces.

The reminder unit can select the optimal reminder method by referring to past reminder history at the time of reminding. For example, the reminder unit selects the optimal reminder method by referring to past reminder history at the time of reminding. Generative AI selects the optimal reminder method by referring to past reminder history. For example, the reminder unit prioritizes reminder methods that have been effective for the user in the past. It can also propose optimal reminder methods for specific time periods based on the user's past reminder history. Furthermore, it can analyze the user's past reminder history and select the most efficient reminder method. Thus, the optimal reminder method can be selected by referring to past reminder history. Specifically, the reminder unit extracts reminder history data accumulated chronologically for each user (e.g., reminder date and time, notification means, notification content, user response (read/unread, action taken or not), notification success rate, etc.) from the database. The reminder unit inputs this history data into a history analysis algorithm using decision trees, random forests, or large language models, and automatically extracts features (e.g., response rate by notification means, action rate by time period, effectiveness by notification content, correlation between notification frequency and behavior promotion, etc.). Examples of AI input include history records such as: “2024-05-01 08:00 push notification success”; “2024-05-02 20:00 email notification unread”; “2024-05-03 07:00 voice notification action taken”. Examples of AI output include: “Recommend voice notification in the morning”; “Avoid email notification at night”; “Increase detailed notifications on weekends”; and priority lists of notification means (e.g., voice notification>push notification>email notification). The reminder unit matches these outputs with the user's current usage status and environmental information to automatically select the optimal reminder method and perform notification/switching on the user interface. Furthermore, the logic for selecting the reminder method is sequentially learned and optimized according to each user's history pattern and changes in notification effectiveness, resulting in greatly improved usability, behavior promotion effect, and notification efficiency compared to conventional uniform notification methods. As a technical effect, the reminder unit analyzes large amounts of history data in a high-dimensional feature space to realize personalization and automatic optimization of reminder methods, enabling flexible adaptation to diverse usage environments and needs for each user. Application fields include health management systems, telemedicine, nursing care settings, corporate health management support, and behavior change support systems.

The reminder unit can customize the timing of reminders based on the user's current health status at the time of reminding. For example, the reminder unit customizes the timing of reminders according to the user's current health status at the time of reminding. Generative AI customizes the timing of reminders based on the user's current health status. For example, when the user's health status is good, normal reminder timing is provided. When the user's health status is deteriorating, earlier reminder timing is provided. Furthermore, when the user's health status is improving, the frequency of reminders is reduced. Thus, the timing of reminders can be customized based on the user's current health status. Specifically, the reminder unit analyzes the user's health status data obtained from the recording unit (e.g., recent diagnosis name, test results, medication history, stress level, sleep score, amount of exercise, etc.) in chronological order. The reminder unit determines the trend of health status changes (e.g., worsening, improving, stable) using machine learning models (e.g., time-series prediction models, decision trees, random forests, etc.) or rule-based algorithms, and automatically determines the reminder timing (e.g., normal, advanced, reduced frequency, etc.). Examples of AI input include health status data such as: “2024-05-01 gastritis diagnosis”; “2024-05-02 blood pressure increase”; “2024-05-03 sleep deprivation”. Examples of AI output include: “Advance examination reminder by one week due to worsening health status”; “Reduce reminder frequency due to improving health status”; “Maintain normal reminder timing”. Based on these outputs, the reminder unit automatically adjusts notification timing and frequency, realizing reminders optimized for the user's health status. Furthermore, the logic for customizing reminder timing is sequentially learned and optimized based on the user's health status trends and feedback from reminder response history, resulting in greatly improved behavior promotion effect, user satisfaction, and health maintenance effect compared to conventional uniform notification methods. As a technical effect, the reminder unit realizes optimization of reminder timing according to health status, supporting health management actions and prevention of examination/treatment delays in medical settings. Application fields include health management systems, telemedicine, nursing care settings, personalized healthcare services, and disease prevention support.

The reminder unit can estimate the user's emotion and determine the priority of reminders based on the estimated emotion. For example, the reminder unit estimates the user's emotion and determines the priority of reminders according to the estimated emotion. Emotion estimation is realized, for example, by using an emotion estimation function with an emotion engine or generative AI. Generative AI may include text generation AI (e.g., LLM) or multimodal generative AI, but is not limited thereto. For example, when the user is feeling stressed, important reminders are prioritized. When the user is relaxed, all reminders are provided equally. Furthermore, when the user is in a hurry, highly urgent reminders are prioritized. Thus, the priority of reminders can be determined based on the user's emotion. Specifically, the reminder unit receives voice input data from the reception unit or recording unit, estimates emotion categories and emotion intensity scores using a voice emotion recognition model (convolutional neural network, recurrent neural network, Transformer, etc.), and combines them with a list of reminder candidates (e.g., examination reminders, medication reminders, lifestyle reminders, etc.) to apply a priority determination algorithm (e.g., weighted scoring, rule-based, machine learning model, etc.). Examples of AI input include: “Emotion estimation: stress level 0.9, reminder candidates: examination, medication, exercise”; “Emotion estimation: relaxation level 0.8, reminder candidates: all items”. Examples of AI output include: “During stress, prioritize only examination reminders”; “During relaxation, notify all reminders equally”; “When in a hurry, notify only highly urgent reminders”. Based on these outputs, the reminder unit automatically adjusts the notification order and notification permission of reminders, prioritizing notifications to the user for reminders with high importance or urgency. Furthermore, the logic for determining priority is sequentially learned and optimized based on the user's emotion estimation history and feedback from reminder response history, resulting in greatly improved emergency response capability, behavior promotion effect, and user satisfaction compared to conventional uniform notification methods. As a technical effect, the reminder unit realizes priority control by combining emotion estimation and content analysis, enabling flexible reminder notifications according to the user's psychological state and situation. Application fields include health management systems, telemedicine, nursing care settings, emergency notification systems, and personalized healthcare services.

The reminder unit can select the optimal reminder method by considering the user's geographic location information at the time of reminding. For example, the reminder unit selects the optimal reminder method by considering the user's geographic location information at the time of reminding. Generative AI selects the optimal reminder method by considering the user's geographic location information. For example, when the user is in a specific region, reminders related to that region are prioritized. When the user is traveling, reminders related to the travel destination can also be prioritized. Furthermore, when the user is at home, reminders related to home can also be prioritized. Thus, the optimal reminder method can be selected by considering the user's geographic location information. Specifically, the reminder unit obtains geographic location data (e.g., latitude/longitude, address, facility name, etc.) in real time from the user's device via GPS, Wi-Fi positioning, base station information, etc., and links it to the reminder candidate list to optimize the notification method. The reminder unit combines voice input content converted to text by a voice recognition model and health status data with geographic information, and applies a natural language processing module (place name extraction, facility name extraction, category classification, etc.) or rule-based algorithm. Examples of AI input include: “Current location: Chiyoda-ku, Tokyo, reminder candidate: gastroscopy”; “Current location: Kita-ku, Osaka, reminder candidate: blood test”; “Current location: home, reminder candidate: medication”. Examples of AI output include: “Prioritize notification of examination reminders at medical institutions in Chiyoda-ku”; “Prioritize notification of health consultation reminders at travel destinations”; “Normal notification of medication reminders related to home”. Based on these outputs, the reminder unit prioritizes reminders with high geographic relevance and notifies reminders with low relevance as pending or low priority. Furthermore, by linking with a geographic information database and dynamically acquiring and utilizing medical institution information and health service information for each region, notification accuracy and convenience are improved. As a technical effect, the reminder unit realizes notification optimization utilizing the user's geographic location information, contributing to improved adaptation to regional health issues and access to medical resources. Compared to conventional uniform notification methods, regional adaptability, user satisfaction, and system efficiency are greatly improved. Application fields include health management systems, telemedicine, regional medical cooperation, and health support services for travelers.

The reminder unit can analyze the user's social media activity at the time of reminding and propose means of reminding. For example, the reminder unit analyzes the user's social media activity at the time of reminding and proposes means of reminding. Generative AI analyzes the user's social media activity and proposes means of reminding. For example, reminders related to topics of interest are prioritized based on the user's social media activity. The reminder unit can also analyze the user's social media activity and propose relevant means of reminding. Furthermore, unnecessary reminders can be filtered based on the user's social media activity. Thus, the reminder unit can analyze the user's social media activity and propose means of reminding. Specifically, the reminder unit periodically collects and accumulates structured data (e.g., post date/time, topic tags, emotion labels, post content text, etc.) such as post history, comments, like history, and follow relationships obtained via API from multiple social media platforms used by the user (e.g., microblogs, photo sharing services, health community sites, etc.). At the time of reminding, the reminder unit inputs this social media activity data into natural language processing algorithms (e.g., BERT-based topic classification model, emotion analysis model, clustering algorithm, etc.), and automatically extracts the user's interest topic distribution (e.g., interest scores for categories such as health, exercise, diet, sleep, stress, etc.) and recent topic trends. Examples of AI input include history data such as: “2024-05-01 # exercise post”; “2024-05-02 # sleep comment”; “2024-05-03 # stress like”; and post text (e.g., “I recently started jogging”, “I have been sleep deprived”). The reminder unit matches reminder candidates (e.g., exercise reminder, sleep reminder, stress care reminder, etc.) with interest topics extracted from social media activity, and examples of AI output include: “Prioritize notification of exercise reminders matching # exercise topic”; “Hold diet-related reminders due to low interest”; “Notify stress-related reminders as high importance”. Based on these outputs, the reminder unit automatically determines notification permission, priority, and means (e.g., SNS-linked notification, in-app notification, email notification, etc.) for reminders, and notifies only highly relevant reminders to the user. Furthermore, the filtering algorithm is sequentially learned and optimized according to changes in the user's social media activity and emergence of new interest topics, resulting in greatly improved notification accuracy, usability, and system efficiency compared to conventional uniform notification methods. As a technical effect, the reminder unit realizes notification filtering adapted to each user's interest topics and behavioral tendencies, preventing unnecessary or erroneous notifications and improving the accuracy of important reminders. Application fields include health management systems, personalized healthcare services, telemedicine, automated record-keeping in nursing care settings, corporate health management support, user life log analysis, and well-being support services.

The suggestion unit can estimate the user's emotion and adjust the suggestion method based on the estimated emotion. For example, the suggestion unit estimates the user's emotion and adjusts the suggestion method according to the estimated emotion. Emotion estimation is realized, for example, by using an emotion estimation function with an emotion engine or generative AI. Generative AI may include text generation AI (e.g., LLM) or multimodal generative AI, but is not limited thereto. For example, when the user is relaxed, detailed suggestions are provided. When the user is in a hurry, concise suggestions are provided. Furthermore, when the user is feeling stressed, only important suggestions are provided. Thus, the suggestion method can be adjusted based on the user's emotion. Specifically, the suggestion unit receives voice input data (e.g., 16 kHz sampled voice waveform, length 80,000 samples) from the reception unit or recording unit, performs feature extraction processing such as spectrogram conversion or Mel-frequency cepstral coefficient (MFCC) extraction, and estimates emotion categories (e.g., relaxed, stressed, tense, angry, etc.) and emotion intensity scores (e.g., stress level 0.8, relaxation level 0.1) using a voice emotion recognition model (convolutional neural network, recurrent neural network, Transformer, etc.). The suggestion unit combines the estimated emotion information with the user's health status, history data, and interest area data, and inputs them into a natural language generation model (large language model, etc.) or rule-based suggestion control algorithm to automatically select the suggestion method (e.g., detailed suggestion, concise suggestion, key point emphasis suggestion, etc.). Examples of AI input include: “Voice input: I am very tired today, emotion estimation: stress level 0.9”; “Voice input: I feel good today, emotion estimation: relaxation level 0.8”. Examples of AI output include: “Detailed suggestion: present examination menu, recommended hospitals, and lifestyle improvement advice in detail”; “Concise suggestion: present only today's recommended items”; “Key point suggestion: emphasize only important items”. Based on these outputs, the suggestion unit automatically controls the granularity and expression style of suggestion content (e.g., detailed explanation, bullet points, summary format, etc.), display order, and emphasis on the user interface. Furthermore, the logic for selecting the suggestion method is sequentially learned and optimized based on the user's emotion estimation history and feedback from suggestion usage (e.g., tendency to view only key points when stressed, tendency to prefer detailed suggestions when relaxed), resulting in greatly improved suggestion acceptance, usability, and satisfaction compared to conventional uniform suggestion methods. As a technical effect, the suggestion unit realizes personalized suggestion optimization by combining emotion estimation and suggestion control, enabling flexible health management support according to the user's psychological state and usage scene. Application fields include health management systems, telemedicine, automated record-keeping in nursing care settings, personalized healthcare services, stress management support, and emotion-adaptive user interfaces.

The suggestion unit can analyze the user's past health data at the time of suggestion and select the optimal suggestion method. For example, the suggestion unit analyzes the user's past health data at the time of suggestion and selects the optimal suggestion method. Generative AI analyzes the user's past health data and selects the optimal suggestion method. For example, the suggestion unit proposes the optimal examination menu based on the user's past health data. It can also analyze the user's past health data and propose appropriate hospitals. Furthermore, it can provide health management advice based on the user's past health data. Thus, the optimal suggestion method can be selected by analyzing the user's past health data. Specifically, the suggestion unit extracts the user's health data (e.g., diagnosis history, test results, medication history, exercise/diet/sleep records, etc. as time-series structured data) obtained from the recording unit from the database, applies time-series analysis models (e.g., LSTM, GRU, Transformer-based time-series prediction models) and feature extraction algorithms to automatically extract trends in health status changes and risk factors (e.g., rising blood pressure, lack of exercise, poor nutritional balance, etc.). The suggestion unit inputs the extracted features and risk scores into a rule-based or large language model-based suggestion generation algorithm to automatically generate optimized examination menus (e.g., gastroscopy, blood test, lifestyle disease screening, etc.), recommended hospital lists (e.g., nearby specialized medical institutions), and health management advice (e.g., review exercise habits, propose dietary improvements, etc.) for each user. Examples of AI input include health history data such as: “2024-05-01 gastritis diagnosis”; “2024-05-02 blood pressure increase”; “2024-05-03 sleep deprivation”; and “Unexamined body parts in the past year: stomach, kidney”. Examples of AI output include: “Recommend gastroscopy”; “Lifestyle improvement advice for blood pressure management”; “Suggest nearest gastroenterology specialist hospital”. The suggestion unit combines these outputs with the user's current location and usage trends to automatically adjust the priority and expression method of suggestion content, and presents suggestions optimized for the user's health issues on the user interface. Furthermore, the logic for selecting the suggestion method is sequentially learned and optimized based on the user's history patterns and feedback from suggestion acceptance history, resulting in greatly improved suggestion accuracy, usability, and health behavior promotion effect compared to conventional uniform suggestion methods. As a technical effect, the suggestion unit analyzes large amounts of health data in a high-dimensional feature space to realize personalization and automatic optimization of suggestion methods, enabling flexible adaptation to diverse health issues and needs for each user. Application fields include health management systems, telemedicine, nursing care settings, corporate health management support, and preventive medicine support systems.

The suggestion unit can customize the content of suggestions based on the user's current health status at the time of suggestion. For example, the suggestion unit customizes the content of suggestions according to the user's current health status at the time of suggestion. Generative AI customizes the content of suggestions based on the user's current health status. For example, when the user's health status is good, normal suggestions are provided. When the user's health status is deteriorating, early examination may be suggested. Furthermore, when the user's health status is improving, suggestions for maintaining health may be provided. Thus, the content of suggestions can be customized based on the user's current health status. Specifically, the suggestion unit analyzes the latest health status data obtained from the recording unit (e.g., recent diagnosis name, test results, medication history, stress level, sleep score, amount of exercise, etc.) in real time, determines the trend of health status changes (e.g., worsening, improving, stable) using machine learning models (e.g., time-series prediction models, decision trees, random forests, etc.) or rule-based algorithms. The suggestion unit inputs the determination result into a natural language generation model (large language model, etc.) or suggestion control algorithm to automatically generate suggestion content (e.g., normal suggestion, early examination suggestion, health maintenance suggestion, etc.). Examples of AI input include health status such as: “Health status: blood pressure increase, sleep deprivation, stress level 0.7”; “Health status: good, sufficient exercise”. Examples of AI output include: “Recommend early examination for blood pressure management”; “Suggest continuation of exercise habits for health maintenance”; “Suggest relaxation methods for sleep improvement”. Based on these outputs, the suggestion unit presents suggestion content optimized for the user's health status on the user interface, and automatically adjusts notification timing and expression method as necessary. Furthermore, the logic for customizing suggestion content is sequentially learned and optimized based on the user's health status trends and feedback from suggestion acceptance history, resulting in greatly improved health behavior promotion effect, user satisfaction, and health maintenance effect compared to conventional uniform suggestion methods. As a technical effect, the suggestion unit realizes optimization of suggestion content according to health status, supporting health management actions and prevention of examination/treatment delays in medical settings. Application fields include health management systems, telemedicine, nursing care settings, personalized healthcare services, and disease prevention support.

The suggestion unit can estimate the user's emotion and determine the priority of suggestions based on the estimated emotion. For example, the suggestion unit estimates the user's emotion and determines the priority of suggestions according to the estimated emotion. Emotion estimation is realized, for example, by using an emotion estimation function with an emotion engine or generative AI. Generative AI may include text generation AI (e.g., LLM) or multimodal generative AI, but is not limited thereto. For example, when the user is feeling stressed, important suggestions are prioritized. When the user is relaxed, all suggestions are provided equally. Furthermore, when the user is in a hurry, highly urgent suggestions are prioritized. Thus, the priority of suggestions can be determined based on the user's emotion. Specifically, the suggestion unit receives voice input data from the reception unit or recording unit, estimates emotion categories and emotion intensity scores using a voice emotion recognition model (convolutional neural network, recurrent neural network, Transformer, etc.), and combines them with a list of suggestion candidates (e.g., examination suggestions, lifestyle improvement suggestions, medical institution guidance, etc.) to apply a priority determination algorithm (e.g., weighted scoring, rule-based, machine learning model, etc.). Examples of AI input include: “Emotion estimation: stress level 0.9, suggestion candidates: examination, lifestyle improvement, exercise”; “Emotion estimation: relaxation level 0.8, suggestion candidates: all items”. Examples of AI output include: “During stress, prioritize only examination suggestions”; “During relaxation, present all suggestions equally”; “When in a hurry, present only highly urgent suggestions”. Based on these outputs, the suggestion unit automatically adjusts the presentation order and permission of suggestions, prioritizing presentation to the user for suggestions with high importance or urgency. Furthermore, the logic for determining priority is sequentially learned and optimized based on the user's emotion estimation history and feedback from suggestion acceptance history, resulting in greatly improved emergency response capability, behavior promotion effect, and user satisfaction compared to conventional uniform suggestion methods. As a technical effect, the suggestion unit realizes priority control by combining emotion estimation and content analysis, enabling flexible suggestion presentation according to the user's psychological state and situation. Application fields include health management systems, telemedicine, nursing care settings, emergency notification systems, and personalized healthcare services.

The suggestion unit can select the optimal suggestion method by considering the user's geographic location information at the time of suggestion. For example, the suggestion unit selects the optimal suggestion method by considering the user's geographic location information at the time of suggestion. Generative AI selects the optimal suggestion method by considering the user's geographic location information. For example, when the user is in a specific region, suggestions related to that region are prioritized. When the user is traveling, suggestions related to the travel destination can also be prioritized. Furthermore, when the user is at home, suggestions related to home can also be prioritized. Thus, the optimal suggestion method can be selected by considering the user's geographic location information. Specifically, the suggestion unit obtains geographic location data (e.g., latitude/longitude, address, facility name, etc.) in real time from the user's device via GPS, Wi-Fi positioning, base station information, etc., and links it to the suggestion candidate list to optimize the suggestion content. The suggestion unit combines voice input content converted to text by a voice recognition model and health status data with geographic information, and applies a natural language processing module (place name extraction, facility name extraction, category classification, etc.) or rule-based algorithm. Examples of AI input include: “Current location: Chiyoda-ku, Tokyo, suggestion candidate: gastroscopy”; “Current location: Kita-ku, Osaka, suggestion candidate: blood test”; “Current location: home, suggestion candidate: lifestyle improvement”. Examples of AI output include: “Prioritize presentation of examination suggestions at medical institutions in Chiyoda-ku”; “Prioritize presentation of health consultation suggestions at travel destinations”; “Normal presentation of lifestyle improvement suggestions related to home”. Based on these outputs, the suggestion unit prioritizes suggestions with high geographic relevance and presents suggestions with low relevance as pending or low priority. Furthermore, by linking with a geographic information database and dynamically acquiring and utilizing medical institution information and health service information for each region, suggestion accuracy and convenience are improved. As a technical effect, the suggestion unit realizes suggestion optimization utilizing the user's geographic location information, contributing to improved adaptation to regional health issues and access to medical resources. Compared to conventional uniform suggestion methods, regional adaptability, user satisfaction, and system efficiency are greatly improved. Application fields include health management systems, telemedicine, regional medical cooperation, and health support services for travelers.

The suggestion unit can analyze the user's social media activity at the time of suggestion and propose means of suggestion. For example, the suggestion unit analyzes the user's social media activity at the time of suggestion and proposes means of suggestion. Generative AI analyzes the user's social media activity and proposes means of suggestion. For example, suggestions related to topics of interest are prioritized based on the user's social media activity. The suggestion unit can also analyze the user's social media activity and propose relevant means of suggestion. Furthermore, unnecessary suggestions can be filtered based on the user's social media activity. Thus, the suggestion unit can analyze the user's social media activity and propose means of suggestion. Specifically, the suggestion unit periodically collects and accumulates structured data (e.g., post date/time, topic tags, emotion labels, post content text, etc.) such as post history, comments, like history, and follow relationships obtained via API from multiple social media platforms used by the user (e.g., microblogs, photo sharing services, health community sites, etc.). At the time of suggestion, the suggestion unit inputs this social media activity data into natural language processing algorithms (e.g., BERT-based topic classification model, emotion analysis model, clustering algorithm, etc.), and automatically extracts the user's interest topic distribution (e.g., interest scores for categories such as health, exercise, diet, sleep, stress, etc.) and recent topic trends. Examples of AI input include history data such as: “2024-05-01 # exercise post”; “2024-05-02 # sleep comment”; “2024-05-03 # stress like”; and post text (e.g., “I recently started jogging”, “I have been sleep deprived”). The suggestion unit matches suggestion candidates (e.g., exercise suggestion, sleep improvement suggestion, stress care suggestion, etc.) with interest topics extracted from social media activity, and examples of AI output include: “Prioritize presentation of exercise suggestions matching # exercise topic”; “Hold diet-related suggestions due to low interest”; “Present stress-related suggestions as high importance”. Based on these outputs, the suggestion unit automatically determines presentation permission, priority, and means (e.g., SNS-linked notification, in-app notification, email notification, etc.) for suggestions, and presents only highly relevant suggestions to the user. Furthermore, the filtering algorithm is sequentially learned and optimized according to changes in the user's social media activity and emergence of new interest topics, resulting in greatly improved suggestion accuracy, usability, and system efficiency compared to conventional uniform suggestion methods. As a technical effect, the suggestion unit realizes suggestion filtering adapted to each user's interest topics and behavioral tendencies, preventing unnecessary or erroneous suggestions and improving the accuracy of important suggestions. Application fields include health management systems, personalized healthcare services, telemedicine, automated record-keeping in nursing care settings, corporate health management support, user life log analysis, and well-being support services.

The system according to the embodiment is not limited to the examples described above and can be variously modified, for example, as follows. Specifically, the system can combine various data acquisition means such as text input, image input, and biosensor data input from wearable devices in addition to voice input. The system can selectively use architectures for voice recognition models and natural language processing models, such as convolutional neural networks, recurrent neural networks, Transformer, self-supervised learning models, and multimodal integration models. Furthermore, as health data analysis algorithms, time-series prediction models, anomaly detection models, clustering models, and reinforcement learning models can be combined to realize health management, advice, reminders, and suggestions optimized for each user. The database configuration can also be selected according to the application, such as relational databases, time-series databases, graph databases, and distributed databases, thereby enhancing the scalability, flexibility, and fault tolerance of the entire system. Examples of AI input include various types of data such as voice, text, image, sensor data, and user attributes, history, and environmental information, and examples of AI output include text data, structured data, health risk prediction, anomaly detection alerts, personalized advice, reminders, and suggestions. These outputs are used by subsequent display units, reminder units, suggestion units, external linkage APIs, etc. As a technical effect, the system flexibly combines diverse data sources, AI models, and database configurations, greatly improving the accuracy, scalability, usability, and system efficiency of health management compared to conventional single-modality and single-algorithm methods. Application fields include personal health management, telemedicine, automated record-keeping in nursing care settings, corporate health management support, preventive medicine, life log analysis, and well-being support services.

The health management system can further include a function to receive the user's meal records via voice input and analyze nutritional balance. For example, when the user says, “I had bread and coffee for breakfast today,” the system records the content and analyzes the nutritional balance. Next, based on the user's meal history, the system can notify the user of risks of nutritional deficiency or excessive intake. For example, notifications such as “You have recently been lacking vitamin C intake” can be provided. The system can also propose healthy meal menus based on the user's meal history. For example, suggestions such as “For your next meal, we recommend eating oranges, which are rich in vitamin C” can be made. Thus, the system can support the user's meal management and contribute to health maintenance. Specifically, the system receives voice input data (e.g., 16 kHz sampled voice waveform) at the reception unit, converts it to text using a voice recognition model (CNN, RNN, Transformer, etc.), and uses a natural language processing module (named entity extraction, food name extraction, quantity estimation algorithm, etc.) to convert meal content into structured data (e.g., food name, quantity, meal time, etc.). The system links with a food database to automatically map nutrient information for each food item (e.g., energy, protein, fat, carbohydrate, vitamins, minerals, etc.) and calculates the overall nutritional balance of the meal (e.g., daily vitamin C intake, excess fat, etc.). Examples of AI input include: “Voice input: today's lunch was salad and chicken”; “Voice input: dinner was ramen”. Examples of AI output include: “Nutritional balance analysis result: vitamin C deficiency, excess fat”; “Healthy meal suggestion: add vegetables next time”. These outputs are used for notifications to the user, input to health advice generation modules, and meal history graph displays. As a technical effect, the system automatically analyzes meal content from voice input and quantitatively evaluates and proposes nutritional balance, greatly improving recording accuracy, convenience, and health behavior promotion effect compared to conventional manual input or self-reporting methods. Application fields include health management systems, personalized healthcare services, telemedicine, automated record-keeping in nursing care settings, corporate health management support, and nutrition guidance support services.

The health management system may further include a function to receive the user's exercise records via voice input and analyze the amount of exercise. For example, when a user says, “I jogged for 30 minutes today,” the system records the content and analyzes the amount of exercise. Next, based on the user's exercise history, the system can notify the user of risks such as lack of exercise or excessive exercise. For example, notifications such as “Recently, your amount of exercise has been insufficient” may be provided. Furthermore, the system can propose appropriate exercise menus based on the user's exercise history. For example, suggestions such as “For your next exercise, it is recommended to include stretching” may be provided. In this way, the system supports the user's exercise management and contributes to maintaining health. Specifically, the system receives voice input data via the reception unit, converts it to text using a voice recognition model, and uses a natural language processing module (such as exercise type extraction, exercise time estimation, calorie consumption calculation algorithms, etc.) to convert the exercise content into structured data (e.g., exercise type, duration, intensity, calories burned, etc.). The system collaborates with an exercise database and user attributes (such as age, gender, weight, etc.) to automatically calculate individually optimized exercise evaluations and risk assessments (e.g., degree of exercise deficiency, excessive exercise risk score, etc.). Examples of AI input include “Voice input: 1 hour walking today” and “Voice input: 20 minutes of strength training,” while examples of AI output include “Exercise analysis result: target not achieved” and “Exercise suggestion: add stretching next time.” These outputs are used for user notifications, exercise history graph displays, and as input to health advice generation modules. As a technical effect, the system automatically analyzes exercise content from voice input and quantitatively evaluates and proposes exercise amounts and risks, greatly improving recording accuracy, convenience, and health behavior promotion compared to conventional manual input or self-reporting methods. Application fields include health management systems, personalized healthcare services, telemedicine, automated recording in nursing care settings, corporate health management support, and exercise guidance support services.

The health management system may further include a function to receive the user's sleep records via voice input and analyze sleep quality. For example, when a user says, “I slept for 6 hours last night,” the system records the content and analyzes sleep quality. Next, based on the user's sleep history, the system can notify the user of risks such as sleep deprivation or excessive sleep. For example, notifications such as “Recently, your sleep time has been insufficient” may be provided. Furthermore, the system can propose appropriate sleep improvement methods based on the user's sleep history. For example, suggestions such as “For your next sleep, it is recommended to use aroma for relaxation” may be provided. In this way, the system supports the user's sleep management and contributes to maintaining health. Specifically, the system receives voice input data via the reception unit, converts it to text using a voice recognition model, and uses a natural language processing module (such as sleep time extraction, sleep quality estimation algorithms, etc.) to convert sleep content into structured data (e.g., bedtime, wake-up time, sleep duration, sleep quality, etc.). The system collaborates with a sleep database and user attributes (such as age, gender, lifestyle habits, etc.) to automatically generate sleep deprivation risk, excessive sleep risk, and sleep improvement suggestions (e.g., relaxation methods before bedtime, sleep environment improvement, etc.). Examples of AI input include “Voice input: only slept 5 hours last night” and “Voice input: recently having trouble falling asleep,” while examples of AI output include “Sleep deprivation risk notification” and “Sleep improvement suggestion: use aroma.” These outputs are used for user notifications, sleep history graph displays, and as input to health advice generation modules. As a technical effect, the system automatically analyzes sleep content from voice input and quantitatively evaluates and proposes sleep quality and risks, greatly improving recording accuracy, convenience, and health behavior promotion compared to conventional manual input or self-reporting methods. Application fields include health management systems, personalized healthcare services, telemedicine, automated recording in nursing care settings, corporate health management support, and sleep guidance support services.

The health management system may further include a function to receive the user's stress level via voice input and support stress management. For example, when a user says, “I felt very stressed at work today,” the system records the content and analyzes the stress level. Next, based on the user's stress history, the system can provide advice for stress reduction. For example, advice such as “Recently, your stress level has increased. It is recommended to take deep breaths to relax” may be provided. Furthermore, the system can propose activities for stress reduction based on the user's stress history. For example, suggestions such as “For your next break, it is recommended to take a walk” may be provided. In this way, the system supports the user's stress management and contributes to maintaining health. Specifically, the system receives voice input data via the reception unit, converts it to text using a voice recognition model, and uses a natural language processing module (such as stress expression extraction, emotion analysis algorithms, etc.) to convert stress content into structured data (e.g., stress factors, stress level, occurrence time, etc.). The system collaborates with a stress history database and user attributes (such as occupation, lifestyle habits, etc.) to automatically generate stress level evaluations and stress reduction advice (e.g., relaxation methods, exercise suggestions, rest suggestions, etc.). Examples of AI input include “Voice input: had many meetings today and got tired” and “Voice input: recently feeling irritated often,” while examples of AI output include “Stress level analysis result: high” and “Stress reduction suggestion: deep breathing, walking.” These outputs are used for user notifications, stress history graph displays, and as input to health advice generation modules. As a technical effect, the system automatically analyzes stress content from voice input and quantitatively evaluates and proposes stress levels and reduction measures, greatly improving recording accuracy, convenience, and health behavior promotion compared to conventional manual input or self-reporting methods. Application fields include health management systems, personalized healthcare services, telemedicine, automated recording in nursing care settings, corporate health management support, and stress management support services.

The health management system may further include a function to estimate the user's emotion and provide health advice based on the estimated emotion. For example, when a user says, “I am very tired today,” the system estimates the emotion and provides appropriate health advice. For example, advice such as “If you are tired, it is recommended to take sufficient rest” may be provided. Furthermore, the system can propose relaxation methods based on the user's emotion. For example, suggestions such as “To relax, it is recommended to take a warm bath” may be provided. In this way, the system provides health advice based on the user's emotion and contributes to maintaining health. Specifically, the system receives voice input data via the reception unit, estimates emotion categories (e.g., fatigue, relaxation, stress, tension, etc.) and emotion intensity scores using a voice emotion recognition model (CNN, RNN, Transformer, etc.), and automatically generates health advice (e.g., rest suggestions, relaxation method suggestions, activity suggestions, etc.) according to the emotional state using a natural language generation model (large language model, etc.) or rule-based advice generation algorithms. Examples of AI input include “Voice input: I am very tired today, emotion estimation: fatigue level 0.9” and “Voice input: feeling good, emotion estimation: relaxation level 0.8,” while examples of AI output include “Rest suggestion: recommend sufficient sleep” and “Relaxation suggestion: recommend bathing.” These outputs are used for user notifications, health advice history displays, and as input to health behavior promotion modules. As a technical effect, the system automatically estimates emotional states from voice input and provides personalized health advice, greatly improving behavior promotion, user satisfaction, and health maintenance compared to conventional uniform advice methods. Application fields include health management systems, personalized healthcare services, telemedicine, automated recording in nursing care settings, corporate health management support, and stress management support services.

The health management system may further include a function to estimate the user's emotion and adjust exercise menus based on the estimated emotion. For example, when a user says, “I am feeling down today,” the system estimates the emotion and proposes an appropriate exercise menu. For example, suggestions such as “If you are feeling down, it is recommended to do light walking” may be provided. Furthermore, the system can adjust the intensity of exercise based on the user's emotion. For example, suggestions such as “You are feeling good today, so jogging is recommended” may be provided. In this way, the system adjusts exercise menus based on the user's emotion and contributes to maintaining health. Specifically, the system receives voice input data via the reception unit, estimates emotion categories and emotion intensity scores using a voice emotion recognition model, and automatically generates exercise menus (e.g., light exercise, normal exercise, high-intensity exercise, etc.) and exercise types (e.g., walking, jogging, stretching, etc.) according to the emotional state using a natural language generation model and exercise suggestion algorithms. Examples of AI input include “Voice input: feeling down today, emotion estimation: negative level 0.8” and “Voice input: feeling good, emotion estimation: positive level 0.9,” while examples of AI output include “Exercise suggestion: light walking” and “Exercise suggestion: jogging.” These outputs are used for user notifications, exercise history displays, and as input to health advice generation modules. As a technical effect, the system automatically estimates emotional states from voice input and proposes personalized exercise menus, greatly improving behavior promotion, user satisfaction, and health maintenance compared to conventional uniform exercise suggestion methods. Application fields include health management systems, personalized healthcare services, telemedicine, automated recording in nursing care settings, corporate health management support, and exercise guidance support services.

The health management system may further include a function to estimate the user's emotion and adjust meal menus based on the estimated emotion. For example, when a user says, “I am very tired today,” the system estimates the emotion and proposes an appropriate meal menu. For example, suggestions such as “If you are tired, it is recommended to have a highly nutritious meal” may be provided. Furthermore, the system can adjust the content of meals based on the user's emotion. For example, suggestions such as “You are feeling good today, so a light salad is recommended” may be provided. In this way, the system adjusts meal menus based on the user's emotion and contributes to maintaining health. Specifically, the system receives voice input data via the reception unit, estimates emotion categories and emotion intensity scores using a voice emotion recognition model, and automatically generates meal menus (e.g., highly nutritious meals, light meals, foods with relaxation effects, etc.) and food types according to the emotional state using a natural language generation model and meal suggestion algorithms. Examples of AI input include “Voice input: very tired today, emotion estimation: fatigue level 0.9” and “Voice input: feeling good, emotion estimation: relaxation level 0.8,” while examples of AI output include “Meal suggestion: highly nutritious meal” and “Meal suggestion: salad.” These outputs are used for user notifications, meal history displays, and as input to health advice generation modules. As a technical effect, the system automatically estimates emotional states from voice input and proposes personalized meal menus, greatly improving behavior promotion, user satisfaction, and health maintenance compared to conventional uniform meal suggestion methods. Application fields include health management systems, personalized healthcare services, telemedicine, automated recording in nursing care settings, corporate health management support, and nutrition guidance support services.

The health management system may further include a function to estimate the user's emotion and adjust the sleep environment based on the estimated emotion. For example, when a user says, “I am very tired today,” the system estimates the emotion and proposes an appropriate sleep environment. For example, suggestions such as “If you are tired, it is recommended to sleep in a quiet environment” may be provided. Furthermore, the system can propose methods to improve sleep quality based on the user's emotion. For example, suggestions such as “You are feeling good today, so it is recommended to use aroma for relaxation” may be provided. In this way, the system adjusts the sleep environment based on the user's emotion and contributes to maintaining health. Specifically, the system receives voice input data via the reception unit, estimates emotion categories and emotion intensity scores using a voice emotion recognition model, and automatically generates sleep environment improvement suggestions (e.g., quiet environment, use of aroma, lighting adjustment, etc.) and pre-sleep activity suggestions according to the emotional state using a natural language generation model and sleep environment suggestion algorithms. Examples of AI input include “Voice input: very tired today, emotion estimation: fatigue level 0.9” and “Voice input: feeling good, emotion estimation: relaxation level 0.8,” while examples of AI output include “Sleep environment suggestion: quiet environment” and “Sleep environment suggestion: use of aroma.” These outputs are used for user notifications, sleep history displays, and as input to health advice generation modules. As a technical effect, the system automatically estimates emotional states from voice input and provides personalized sleep environment suggestions, greatly improving behavior promotion, user satisfaction, and health maintenance compared to conventional uniform suggestion methods. Application fields include health management systems, personalized healthcare services, telemedicine, automated recording in nursing care settings, corporate health management support, and sleep guidance support services.

The health management system may further include a function to estimate the user's emotion and propose stress management methods based on the estimated emotion. For example, when a user says, “I am feeling very stressed today,” the system estimates the emotion and proposes appropriate stress management methods. For example, suggestions such as “If you are feeling stressed, it is recommended to take deep breaths” may be provided. Furthermore, the system can propose activities for stress reduction based on the user's emotion. For example, suggestions such as “You are feeling good today, so it is recommended to do yoga” may be provided. In this way, the system proposes stress management methods based on the user's emotion and contributes to maintaining health. Specifically, the system receives voice input data via the reception unit, estimates emotion categories and emotion intensity scores using a voice emotion recognition model, and automatically generates stress reduction methods (e.g., deep breathing, walking, yoga, meditation, etc.) and activity suggestions according to the emotional state using a natural language generation model and stress management suggestion algorithms. Examples of AI input include “Voice input: feeling very stressed today, emotion estimation: stress level 0.9” and “Voice input: feeling good, emotion estimation: relaxation level 0.8,” while examples of AI output include “Stress management suggestion: deep breathing” and “Stress management suggestion: yoga.” These outputs are used for user notifications, stress history displays, and as input to health advice generation modules. As a technical effect, the system automatically estimates emotional states from voice input and proposes personalized stress management methods, greatly improving behavior promotion, user satisfaction, and health maintenance compared to conventional uniform suggestion methods. Application fields include health management systems, personalized healthcare services, telemedicine, automated recording in nursing care settings, corporate health management support, and stress management support services.

The health management system may further include a function to provide advice for preventive medicine based on the user's health data. For example, the system analyzes the user's past health data and predicts diseases that may pose a higher risk in the future. Next, based on the prediction, the system provides advice for preventive medicine. For example, advice such as “There is a possibility that your risk of hypertension may increase in the future, so it is recommended to reduce salt intake” may be provided. Furthermore, the system can notify the user of the importance of regular health checks based on the user's health data. For example, notifications such as “It is recommended to measure your blood pressure regularly” may be provided. In this way, the system provides advice for preventive medicine based on the user's health data and contributes to maintaining health. Specifically, the system obtains health data (e.g., diagnosis history, test results, lifestyle data, vital data, etc.) from the recording unit, analyzes it using chronological analysis models (LSTM, GRU, Transformer, etc.) and anomaly detection models, and automatically predicts the probability of future health risks (e.g., hypertension, diabetes, heart disease, etc.) and risk scores. The system uses the prediction results as input and automatically generates preventive medicine advice (e.g., dietary restrictions, exercise habits, recommendations for periodic examinations, etc.) and health check notifications using a natural language generation model or rule-based advice generation algorithms. Examples of AI input include “Blood pressure data for the past year” and “Lifestyle: lack of exercise, excessive salt intake,” while examples of AI output include “Hypertension risk prediction: high,” “Preventive advice: salt restriction,” and “Health check notification: recommend regular blood pressure measurement.” These outputs are used for user notifications, health advice history displays, and as input to health behavior promotion modules. As a technical effect, the system automatically predicts future risks from health data and provides personalized preventive medicine advice, greatly improving preventive effects, user satisfaction, and health maintenance compared to conventional uniform advice methods. Application fields include health management systems, personalized healthcare services, telemedicine, automated recording in nursing care settings, corporate health management support, and preventive medicine support services.

Below, the processing flow of Example of the Embodiment is briefly described. Specifically, the system adopts a configuration in which multiple functional modules such as a reception unit, a recording unit, a display unit, a reminder unit, and a suggestion unit operate in cooperation. Each module exchanges voice input data, structured data, user attributes, history data, environmental information, etc., and executes processing by combining AI models and rule-based algorithms. The data flow includes a series of processes: receiving voice input, voice recognition and conversion to text, content analysis and structuring, storage in a history database, chronological display, reminder generation, suggestion generation, presentation via user interface, acquisition of user feedback, and sequential learning and optimization of AI models. Examples of AI input include voice waveform data, text data, health history, emotion estimation values, and environmental information, while examples of AI output include text data, structured data, reminder content, suggestion content, and display control parameters. These outputs are used in subsequent modules and user interfaces to realize the overall health management support function of the system. As a technical effect, the system integrally links multiple AI models, algorithms, databases, and user interfaces, greatly improving the accuracy, efficiency, usability, and scalability of health management compared to conventional single-function systems. Application fields include health management systems, telemedicine, automated recording in nursing care settings, corporate health management support, personalized healthcare services, and preventive medicine support services.

Step 1: The reception unit receives content spoken by a user via voice. For example, when a user says, “I was diagnosed with gastritis at the hospital,” the reception unit receives the content. Step 2: The recording unit analyzes the voice received by the reception unit and records it as text data. For example, a generative AI converts the voice to text data using a voice recognition algorithm and records the content. Step 3: The display unit displays the data recorded by the recording unit in chronological order. For example, the user can check a list of which hospital they visited, what diagnosis they received, which medication they took and when, which examination they underwent and when, and the results. Step 4: The reminder unit provides reminders when periodic examinations are required. For example, by providing reminders such as “The next blood test is next month,” the user is prevented from forgetting the examination. Step 5: The suggestion unit indicates body parts that have not been examined recently and proposes appropriate human health checkup menus and nearby hospitals. For example, suggestions such as “You have not had a stomach examination recently, so it is recommended to undergo a gastroscopy. You can have it at a nearby hospital” may be provided. Specifically, in Step 1, the system receives voice input data (e.g., 16 kHz sampled voice waveform) via the reception unit, performs preprocessing such as spectrogram conversion and MFCC extraction, and converts it to text using a voice recognition model (CNN, RNN, Transformer, etc.). In Step 2, the recording unit analyzes the text data using a natural language processing module (morphological analysis, named entity extraction, topic classification, etc.), generates structured data such as diagnosis name, hospital name, date, etc., and stores it in a database. In Step 3, the display unit extracts structured data in chronological order and displays it on the user interface in calendar format, timeline format, graph format, etc. In Step 4, the reminder unit calculates the next examination date and notification timing based on examination history, recommended intervals, user attributes, etc., and provides reminders via push notifications, email, etc. In Step 5, the suggestion unit analyzes examination history, user attributes, geographic information, etc., and generates individually optimized examination suggestions and medical institution guidance using gap analysis and large language models for natural language generation, presenting them on the user interface. Examples of AI input include voice waveform data, text data, health history, emotion estimation values, and environmental information, while examples of AI output include text data, structured data, reminder content, suggestion content, and display control parameters. These outputs are linked between modules to realize the overall health management support function of the system. As a technical effect, the system integrally links AI models, algorithms, databases, and user interfaces at each step, greatly improving the accuracy, efficiency, usability, and scalability of health management compared to conventional manual recording, notification, and suggestion methods. Application fields include health management systems, telemedicine, automated recording in nursing care settings, corporate health management support, personalized healthcare services, and preventive medicine support services.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned reception unit, recording unit, display unit, reminder unit, and suggestion unit is implemented by at least one of, for example, the smart device 14 and the data processing apparatus 12. For example, the reception unit receives the user's voice using the microphone 38B of the smart device 14 and generates voice data by the control unit 46A. The recording unit is implemented, for example, by a specific processing unit 290 of the data processing apparatus 12, analyzes the voice data, and records it as text data. The display unit displays the recorded data in chronological order using the display 40A of the smart device 14. The reminder unit is implemented, for example, by the specific processing unit 290 of the data processing apparatus 12 and provides reminders for periodic examinations. The suggestion unit is implemented, for example, by the specific processing unit 290 of the data processing apparatus 12 and proposes appropriate examination menus and hospitals. The correspondence between each unit and the device or control unit is not limited to the examples described above and various modifications are possible.

### Second Embodiment

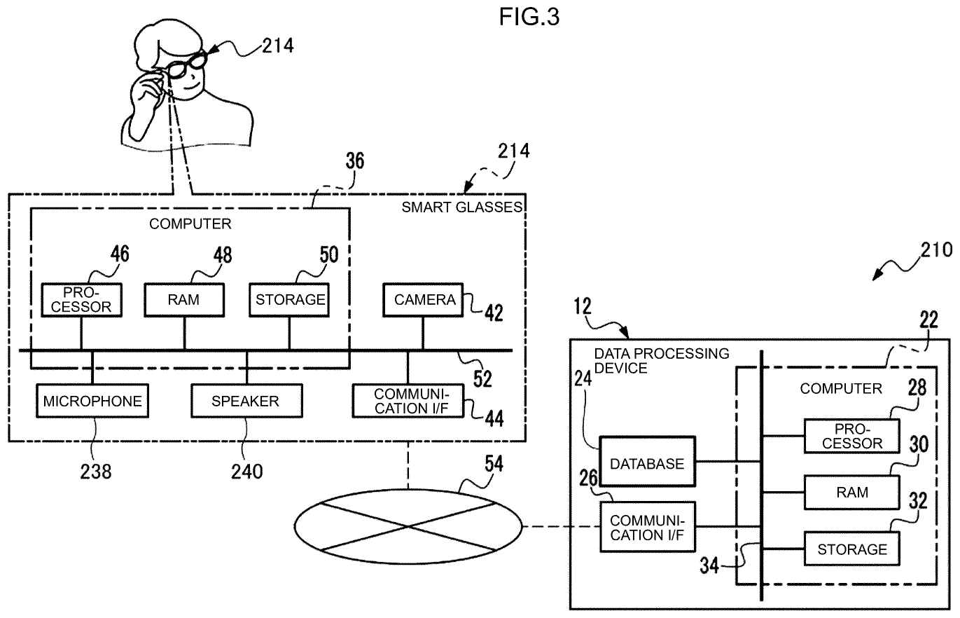

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

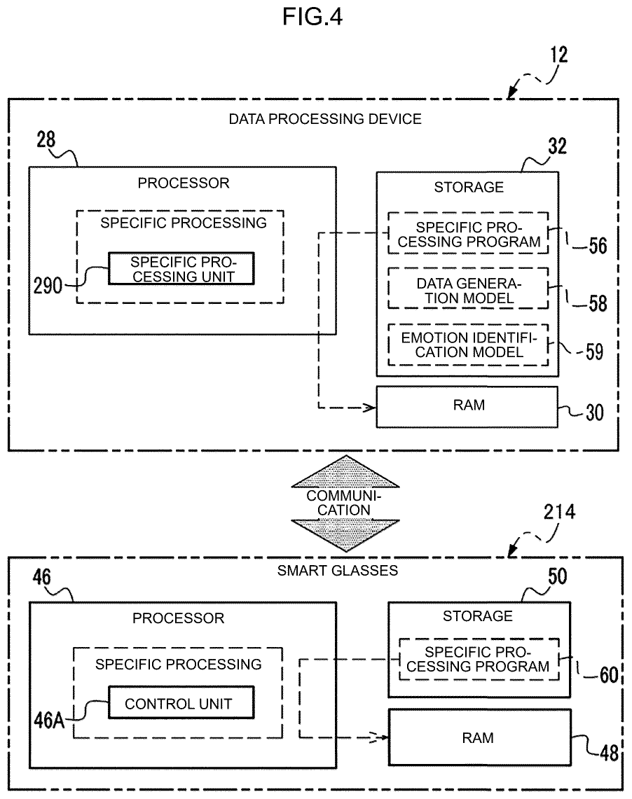

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned reception unit, recording unit, display unit, reminder unit, and suggestion unit is implemented by at least one of, for example, the smart glasses 214 and the data processing apparatus 12. For example, the reception unit receives the user's voice using the microphone 238 of the smart glasses 214 and generates voice data by the control unit 46A. The recording unit is implemented, for example, by a specific processing unit 290 of the data processing apparatus 12, analyzes the voice data, and records it as text data. The display unit displays the recorded data in chronological order using the display of the smart glasses 214. The reminder unit is implemented, for example, by the specific processing unit 290 of the data processing apparatus 12 and provides reminders for periodic examinations. The suggestion unit is implemented, for example, by the specific processing unit 290 of the data processing apparatus 12 and proposes appropriate examination menus and hospitals. The correspondence between each unit and the device or control unit is not limited to the examples described above and various modifications are possible.

### Third Embodiment

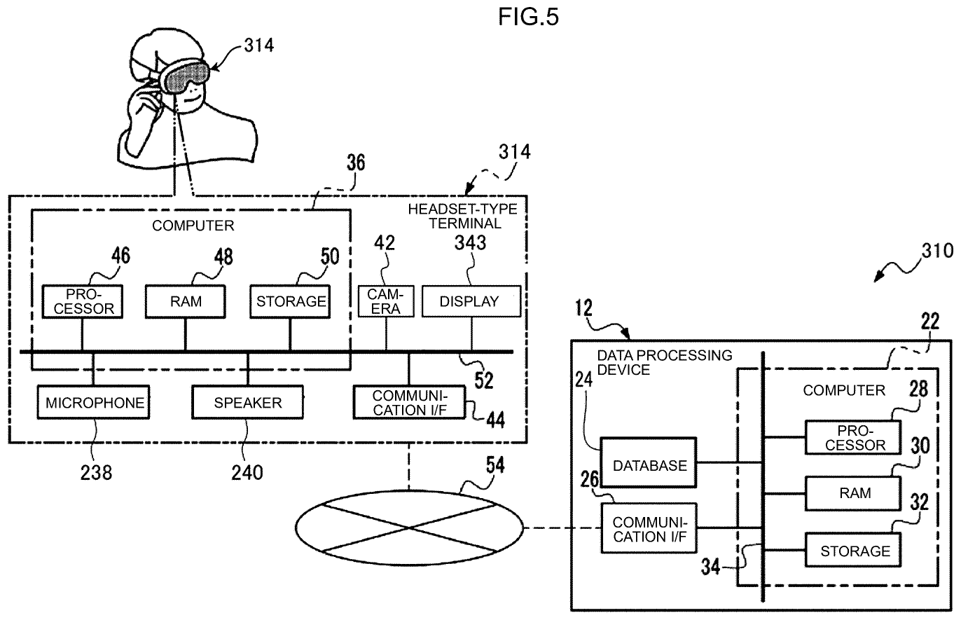

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

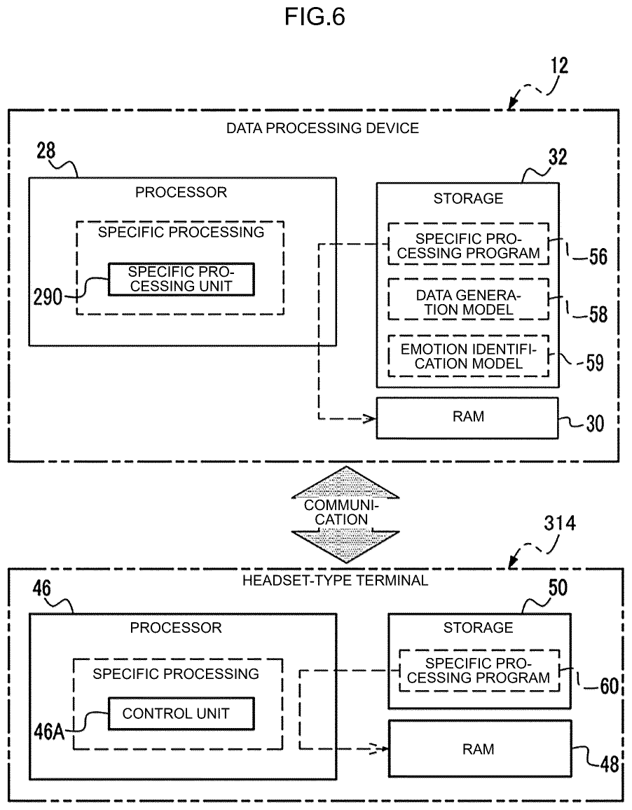

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned reception unit, recording unit, display unit, reminder unit, and suggestion unit is implemented by at least one of, for example, the headset-type terminal 314 and the data processing apparatus 12. For example, the reception unit receives the user's voice using the microphone 238 of the headset-type terminal 314 and generates voice data by the control unit 46A. The recording unit is implemented, for example, by a specific processing unit 290 of the data processing apparatus 12, analyzes the voice data, and records it as text data. The display unit displays the recorded data in chronological order using the display 343 of the headset-type terminal 314. The reminder unit is implemented, for example, by the specific processing unit 290 of the data processing apparatus 12 and provides reminders for periodic examinations. The suggestion unit is implemented, for example, by the specific processing unit 290 of the data processing apparatus 12 and proposes appropriate examination menus and hospitals. The correspondence between each unit and the device or control unit is not limited to the examples described above and various modifications are possible.

### Fourth Embodiment

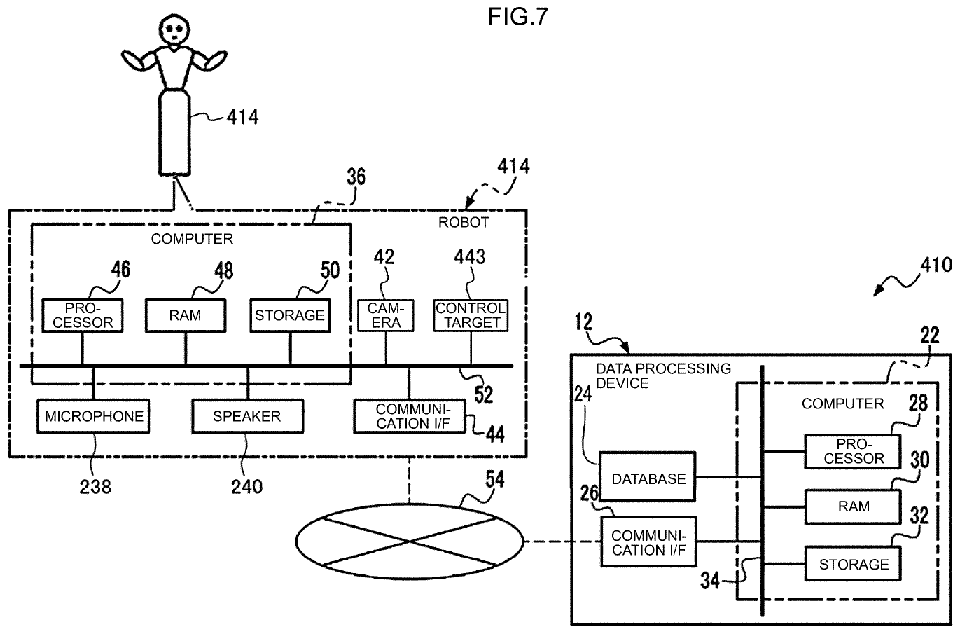

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

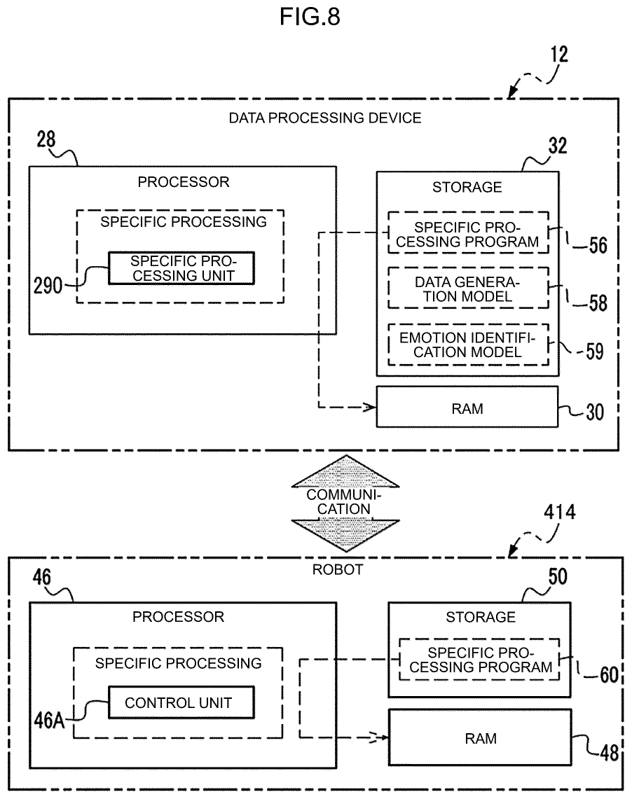

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned reception unit, recording unit, display unit, reminder unit, and suggestion unit is implemented by at least one of, for example, the robot 414 and the data processing apparatus 12. For example, the reception unit receives the user's voice using the microphone 238 of the robot 414 and generates voice data by the control unit 46A. The recording unit is implemented, for example, by a specific processing unit 290 of the data processing apparatus 12, analyzes the voice data, and records it as text data. The display unit displays the recorded data in chronological order using the display of the robot 414. The reminder unit is implemented, for example, by the specific processing unit 290 of the data processing apparatus 12 and provides reminders for periodic examinations. The suggestion unit is implemented, for example, by the specific processing unit 290 of the data processing apparatus 12 and proposes appropriate examination menus and hospitals. The correspondence between each unit and the device or control unit is not limited to the examples described above and various modifications are possible.

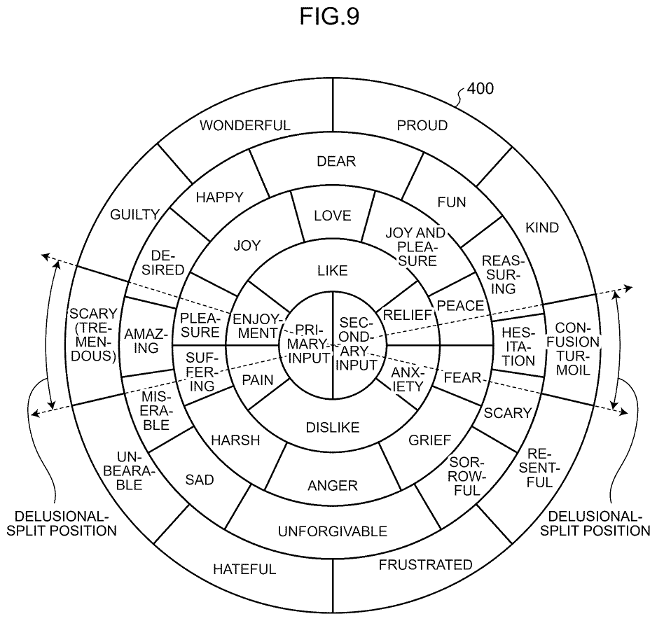

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

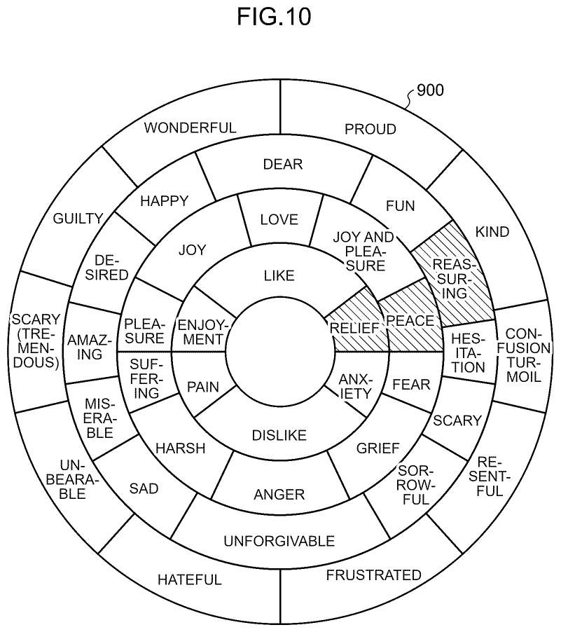

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1)A system comprising: a reception unit configured to receive voice input; a recording unit configured to analyze the voice received by the reception unit and record it as text data; a display unit configured to display the data recorded by the recording unit in chronological order; a reminder unit configured to provide reminders for periodic examinations; and a suggestion unit configured to indicate body parts that have not been examined recently and propose human health checkup menus and nearby hospitals.
(Supplementary Note 2)The system according to Supplementary Note 1, wherein the reception unit is configured to receive content spoken by a user via voice.
(Supplementary Note 3)The system according to Supplementary Note 1, wherein the recording unit is configured to analyze the voice received by the reception unit and record it as text data.
(Supplementary Note 4)The system according to Supplementary Note 1, wherein the display unit is configured to display the data recorded by the recording unit in chronological order.
(Supplementary Note 5)The system according to Supplementary Note 1, wherein the reminder unit is configured to provide reminders when periodic examinations are required.
(Supplementary Note 6)The system according to Supplementary Note 1, wherein the suggestion unit is configured to indicate body parts that have not been examined recently and propose appropriate human health checkup menus and nearby hospitals.
(Supplementary Note 7)The system according to Supplementary Note 1, wherein the reception unit is configured to estimate the user's emotion and adjust the timing of receiving voice input based on the estimated emotion.
(Supplementary Note 8)The system according to Supplementary Note 1, wherein the reception unit is configured to analyze the user's past voice input history and select a reception method.
(Supplementary Note 9)The system according to Supplementary Note 1, wherein the reception unit is configured to perform filtering based on the user's current health status and areas of interest when receiving voice input.
(Supplementary Note 10)The system according to Supplementary Note 1, wherein the reception unit is configured to estimate the user's emotion and determine the priority of voice input to be received based on the estimated emotion.
(Supplementary Note 11)The system according to Supplementary Note 1, wherein the reception unit is configured to prioritize highly relevant information by considering the user's geographic location when receiving voice input.
(Supplementary Note 12)The system according to Supplementary Note 1, wherein the reception unit is configured to analyze the user's social media activity and receive relevant information when receiving voice input.
(Supplementary Note 13)The system according to Supplementary Note 1, wherein the recording unit is configured to estimate the user's emotion and adjust the method of expression in the record based on the estimated emotion.
(Supplementary Note 14)The system according to Supplementary Note 1, wherein the recording unit is configured to adjust the level of detail in the record based on the importance of the voice input at the time of recording.
(Supplementary Note 15)The system according to Supplementary Note 1, wherein the recording unit is configured to apply different recording algorithms according to the category of the voice input at the time of recording.
(Supplementary Note 16)The system according to Supplementary Note 1, wherein the recording unit is configured to estimate the user's emotion and adjust the length of the record based on the estimated emotion.
(Supplementary Note 17)The system according to Supplementary Note 1, wherein the recording unit is configured to determine the priority of the record based on the submission timing of the voice input at the time of recording.
(Supplementary Note 18)The system according to Supplementary Note 1, wherein the recording unit is configured to adjust the order of the record based on the relevance of the voice input at the time of recording.
(Supplementary Note 19)The system according to Supplementary Note 1, wherein the display unit is configured to estimate the user's emotion and adjust the display method based on the estimated emotion.
(Supplementary Note 20)The system according to Supplementary Note 1, wherein the display unit is configured to adjust the level of detail in the display based on the importance of the recorded data at the time of display.
(Supplementary Note 21)The system according to Supplementary Note 1, wherein the display unit is configured to apply different display algorithms according to the category of the data at the time of display.
(Supplementary Note 22)The system according to Supplementary Note 1, wherein the display unit is configured to estimate the user's emotion and adjust the length of the display based on the estimated emotion.
(Supplementary Note 23)The system according to Supplementary Note 1, wherein the display unit is configured to determine the priority of the display based on the submission timing of the data at the time of display.
(Supplementary Note 24)The system according to Supplementary Note 1, wherein the display unit is configured to adjust the order of the display based on the relevance of the data at the time of display.
(Supplementary Note 25)The system according to Supplementary Note 1, wherein the reminder unit is configured to estimate the user's emotion and adjust the reminder method based on the estimated emotion.
(Supplementary Note 26)The system according to Supplementary Note 1, wherein the reminder unit is configured to select a reminder method by referring to past reminder history at the time of reminding.
(Supplementary Note 27)The system according to Supplementary Note 1, wherein the reminder unit is configured to customize the timing of reminders based on the user's current health status at the time of reminding.
(Supplementary Note 28)The system according to Supplementary Note 1, wherein the reminder unit is configured to estimate the user's emotion and determine the priority of reminders based on the estimated emotion.
(Supplementary Note 29)The system according to Supplementary Note 1, wherein the reminder unit is configured to select a reminder method by considering the user's geographic location at the time of reminding.
(Supplementary Note 30)The system according to Supplementary Note 1, wherein the reminder unit is configured to analyze the user's social media activity and propose means of reminding at the time of reminding.
(Supplementary Note 31)The system according to Supplementary Note 1, wherein the suggestion unit is configured to estimate the user's emotion and adjust the suggestion method based on the estimated emotion.
(Supplementary Note 32)The system according to Supplementary Note 1, wherein the suggestion unit is configured to analyze the user's past health data and select a suggestion method at the time of suggestion.
(Supplementary Note 33)The system according to Supplementary Note 1, wherein the suggestion unit is configured to customize the content of suggestions based on the user's current health status at the time of suggestion.
(Supplementary Note 34)The system according to Supplementary Note 1, wherein the suggestion unit is configured to estimate the user's emotion and determine the priority of suggestions based on the estimated emotion.
(Supplementary Note 35)The system according to Supplementary Note 1, wherein the suggestion unit is configured to select a suggestion method by considering the user's geographic location at the time of suggestion.
(Supplementary Note 36)The system according to Supplementary Note 1, wherein the suggestion unit is configured to analyze the user's social media activity and propose means of suggestion at the time of suggestion.

What is claimed is:

1. A system comprising:
circuitry configured to:
receive, via a communication interface coupled to a packet-switched network, an audio signal from a client terminal;
convert the audio signal into a character string by extracting a feature vector from the audio signal and inputting the feature vector into a speech recognition model based on a Transformer architecture;
transmit, to the client terminal via the communication interface, display data representing the character string arranged in a chronological sequence;
generate, by applying a time-series analysis model to a plurality of stored character strings, a notification signal indicating a time interval derived from a recurrence pattern detected across the plurality of stored character strings, and transmit the notification signal to the client terminal via the communication interface; and
generate, by inputting the plurality of stored character strings into a classification model comprising a neural network to identify one or more data categories having no corresponding entries within a threshold time window, a query signal directed to an external server via the packet-switched network to retrieve location-indexed data records associated with the one or more data categories, and transmit response data comprising the location-indexed data records to the client terminal via the communication interface.

2. The system according to claim 1, wherein the circuitry is further configured to extract the feature vector by computing a Mel-frequency cepstral coefficient representation of the audio signal, the feature vector comprising a multidimensional tensor of cepstral coefficients across a plurality of time frames.

3. The system according to claim 1, wherein the circuitry is further configured to apply a named entity recognition model to the character string to extract entity labels and attribute values, and store the entity labels and attribute values as structured data records in a database indexed by a timestamp.

4. The system according to claim 1, wherein the circuitry is further configured to estimate an emotion of a user by inputting the feature vector into an emotion classification model comprising a convolutional neural network, the emotion classification model outputting a probability distribution over a plurality of emotion categories, and adjust a reception timing parameter for the audio signal based on the probability distribution.

5. The system according to claim 1, wherein the circuitry is further configured to analyze a reception history database comprising input device identifiers, timestamps, and recognition accuracy scores for the client terminal, and select a reception method for the audio signal based on a history analysis model applied to the reception history database.

6. The system according to claim 1, wherein the circuitry is further configured to receive user profile data from the database, the user profile data comprising attribute tags and interest category identifiers, and filter the character string by computing a relevance score between the character string and the user profile data using a topic classification model, such that the circuitry stores only character strings having a relevance score exceeding a relevance threshold.

7. The system according to claim 1, wherein the circuitry is further configured to estimate an emotion of a user based on the audio signal and adjust an expression parameter of the character string based on the estimated emotion, the expression parameter comprising at least one of a summarization level, a detail level, or a formatting template.

8. The system according to claim 1, wherein the circuitry is further configured to classify the character string into a content category using a text classification model and apply a recording algorithm selected from a plurality of recording algorithms based on the content category, each recording algorithm defining a different structuring rule for the character string.

9. The system according to claim 1, wherein the circuitry is further configured to compute a relevance score for each character string by inputting the character string and a history of previously stored character strings into a scoring model, and determine a storage priority for the character string based on the relevance score, the storage priority being one of priority, normal, or deferred.

10. The system according to claim 1, wherein the circuitry is further configured to estimate an emotion of a user based on the audio signal and adjust a display parameter of the display data based on the estimated emotion, such that when the estimated emotion indicates a first emotion category, the circuitry generates the display data in a simplified format, and when the estimated emotion indicates a second emotion category, the circuitry generates the display data in a detailed format.

11. The system according to claim 1, wherein the circuitry is further configured to apply different display rendering algorithms according to a data category of the stored character strings, the display rendering algorithms defining at least one of a timeline format, a calendar format, or a graphical chart format.

12. The system according to claim 1, wherein the time-series analysis model comprises at least one of a decision tree model, a random forest model, or a recurrent neural network, and the circuitry determines the time interval by computing a recommended recurrence period from the recurrence pattern and a calendar data set received from the client terminal.

13. The system according to claim 1, wherein the circuitry is further configured to estimate an emotion of a user based on the audio signal and adjust a delivery timing of the notification signal based on the estimated emotion, such that when the estimated emotion indicates stress, the delivery timing is deferred, and when the estimated emotion indicates a neutral state, the delivery timing is advanced.

14. The system according to claim 1, wherein the circuitry is further configured to receive geographic location data from the client terminal and select a notification delivery method from a plurality of delivery methods based on the geographic location data, the plurality of delivery methods comprising at least one of a push notification, an electronic mail message, or a voice announcement transmitted via the communication interface.

15. The system according to claim 1, wherein the circuitry is further configured to receive user attribute data from the database, the user attribute data comprising at least one of an age value, a gender identifier, or a history record, and input the user attribute data together with the one or more data categories into a language generation model to generate a suggestion character string, the response data further comprising the suggestion character string.

16. The system according to claim 1, wherein the circuitry is further configured to receive social media activity data from an external application server via the packet-switched network, and adjust at least one of a reception priority for the audio signal or a content of the response data based on the social media activity data.

17. The system according to claim 1, wherein the circuitry is further configured to preprocess the audio signal by applying at least one of spectral subtraction or a Wiener filter for noise reduction, and applying volume normalization to the audio signal before extracting the feature vector.

18. A system comprising:
a communication interface coupled to a packet-switched network and configured to receive an audio signal from a client terminal and to transmit data to the client terminal;
a memory storing a speech recognition model based on a Transformer architecture, a time-series analysis model, and a classification model comprising a neural network; and
a processor coupled to the communication interface and the memory, the processor configured to:
convert the audio signal into a character string by extracting a feature vector from the audio signal and inputting the feature vector into the speech recognition model;
transmit, to the client terminal via the communication interface, display data representing the character string arranged in a chronological sequence;
generate, by applying the time-series analysis model to a plurality of stored character strings, a notification signal indicating a time interval derived from a recurrence pattern detected across the plurality of stored character strings, and transmit the notification signal to the client terminal via the communication interface; and
generate, by inputting the plurality of stored character strings into the classification model to identify one or more data categories having no corresponding entries within a threshold time window, a query signal directed to an external server via the packet-switched network to retrieve location-indexed data records associated with the one or more data categories, and transmit response data comprising the location-indexed data records to the client terminal via the communication interface.

19. The system according to claim 18, wherein the memory further stores an emotion classification model, and the processor is further configured to estimate an emotion of a user by inputting the feature vector into the emotion classification model to output a probability distribution over a plurality of emotion categories, and adjust at least one of a reception timing parameter for the audio signal or a display parameter of the display data based on the probability distribution.

20. A method performed by a system comprising circuitry, the method comprising:
receiving, via a communication interface coupled to a packet-switched network, an audio signal from a client terminal;
converting the audio signal into a character string by extracting a feature vector from the audio signal and inputting the feature vector into a speech recognition model based on a Transformer architecture;
transmitting, to the client terminal via the communication interface, display data representing the character string arranged in a chronological sequence;
generating, by applying a time-series analysis model to a plurality of stored character strings, a notification signal indicating a time interval derived from a recurrence pattern detected across the plurality of stored character strings, and transmitting the notification signal to the client terminal via the communication interface; and
generating, by inputting the plurality of stored character strings into a classification model comprising a neural network to identify one or more data categories having no corresponding entries within a threshold time window, a query signal directed to an external server via the packet-switched network to retrieve location-indexed data records associated with the one or more data categories, and transmitting response data comprising the location-indexed data records to the client terminal via the communication interface.
